(19) United States
(12) Patent Application Publication
(10) Pub. No.: US 2011/0039788 A1
(43) Pub. Date: Feb. 17, 2011

(54) COMPOSITIONS, METHODS AND KITS FOR DETECTING AND TREATING CANCER

(75) Inventor: Karoline J. Briegel, Miami, FL (US)

Correspondence Address: Gregory A. Nelson;Novak Druce & Quigg LLP, 525 Okeechobee Blvd, Suite 1500, West Palm Beach, FL, 33401, US

(73) Assignee: University of Miami, Miami, FL (US)

(21) Appl. No.: 12/834,303

(22) Filed: Jul. 12, 2010

Related U.S. Application Data
(60) Provisional application No. 61/224,701, filed on Jul. 10, 2009.
(60) Provisional application No. 61/356,317, filed on Jun. 18, 2010.

(51) Int. Cl. A61K 38/16; A61K 31/7105; C12N 5/09; C12Q 1/68; G01N 33/53; A61P 35/00

(52) U.S. Cl. 514/19.4; 514/44 A; 435/325; 435/6; 435/7.21

(57) ABSTRACT

Compositions, kits and methods for inhibiting cancer cell (e.g., breast cancer cell) growth and treating a subject with cancer (e.g., breast cancer) include a therapeutically effective amount of an LBH inhibitor for inhibiting cancer cell growth and a pharmaceutically acceptable carrier, and/or a therapeutically effective amount of Wnt7a protein or nucleic acids encoding Wnt7a protein for inhibiting cancer cell growth and a pharmaceutically acceptable carrier. Methods of treating a subject having cancer (e.g., estrogen receptor negative basal-type breast cancer) include administering to the subject a composition including a pharmaceutical carrier and at least one of: an LBH inhibitor, a WNT7a protein, and a nucleic acid encoding WNT7a protein in an amount effective for inhibiting growth of cancer cells in the subject. Methods of detecting the presence of cancer (e.g., estrogen receptor negative basal-type breast cancer) in a subject include obtaining a biological sample from the subject; contacting the sample with at least one reagent that detects presence of LBH expression; measuring the level of LBH expression in the biological sample; and correlating overexpression of LBH with the presence of cancer (e.g., estrogen receptor negative basal-type breast cancer) in the subject. Kits for detecting the presence of basal-type breast cancer in a subject include at least one reagent for detecting the presence of LBH expression in a biological sample from the subject and instructions for use.

### CROSS-REFERENCE TO RELATED APPLICATIONS

This application claims the benefit of Provisional Application Ser. No. 61/224,701 filed Jul. 10, 2009, and Provisional Application Ser. No. 61/356,317 filed Jun. 18, 2010, which are herein incorporated by reference in their entirety.

### FEDERALLY SPONSORED RESEARCH

This invention was made with government support under grant no. 05-NIR-01-5186 awarded by The Florida State Department of Health. The U.S. government has certain rights in the invention.

### FIELD OF THE INVENTION

The invention relates generally to the fields of molecular genetics, molecular biology, and oncology.

### BACKGROUND

In the United States, cancer is responsible for 25% of all deaths. Death from cancer is primarily due to metastasis of cancer cells to other organs followed by secondary tumor formation throughout the body. Breast cancer, for example, is the second-most common cause of mortality among women with approximately 40,000 women and 480 men newly affected with this disease every year. Despite improved treatment options, breast cancer remains a devastating illness. The most lethal and treatment refractory form of breast cancer is a highly aggressive subtype called “Triple-negative Breast Cancer”. Triple-negative Breast Cancers lack expression of established molecular targets (estrogen receptor/ER, progesterone receptor/PR, HER2 oncogene/ERBB2) that are currently used in the clinic for specific breast cancer treatments based on anti-hormone or herceptin therapies. Moreover, initially good prognosis ER-positive breast cancers can progress into metastatic ER-negative disease, which no longer is curable. Thus, there is a critical need for new specific breast cancer treatments based on molecularly-targeted therapies.

In recent years, cancer stem cells (CSCs), cancer cells that possess characteristics associated with normal stem cells (specifically the ability to give rise to all cell types found in a particular cancer sample), have been shown to play a role in some cancers. CSCs may generate tumors through the stem cell processes of self-renewal and differentiation into multiple cell types, and such cells are proposed to persist in tumors as a distinct population and cause relapse and metastasis by giving rise to new tumors. CSCs are enriched in poor prognosis Triple-negative breast cancers and in metastatic breast disease. Recent studies have shown that CSCs are highly metastatic and have an increased resistance to conventional therapies (radiation and chemo therapy), emphasizing the need for new cancer therapies that specifically target this type of tumor cell. Although great advances have been made towards elucidating the mechanisms of tumorigenesis and metastasis, a need still exists for reagents and methods for treating cancer, including eliminating cancer-initiating CSCs.

### SUMMARY

Described herein are compositions, methods and kits for detecting and treating cancer, for example, breast cancer. It was discovered that LBH is a direct transcriptional target of the WNT/β-catenin pathway, emphasizing the importance of LBH in intrinsic stem/progenitor cell control. Abnormal expression of LBH in WNT-induced mammary tumors in mice, as well as in a highly invasive subtype of human breast cancer (triple-negative breast cancers, a highly metastatic form of breast cancer that is difficult to treat), suggests an important role of LBH in tumorigenesis. In addition, the experiments described herein uncovered an important role of LBH in breast cancer stem cell development. LBH was found to be exclusively expressed in human breast carcinoma cell lines that have a high contribution of CD44^{high}/CD24^{low} or ALDH+ stem cell populations. RNAi-mediated knockdown of LBH in human breast carcinoma cells enriched for CD44^{high}/CD24^{low} cancer stem cell drastically reduced the abundance of the CD44^{high}/CD24^{low} population and resulted in a more differentiated tumor type, suggesting that LBH may be required for cancer stem cell maintenance. Additionally, RNAi-mediated LBH inhibition resulted in marked breast tumor cell death, indicating that LBH is furthermore required for the survival of cancer stem cells. Conversely, ectopic expression of LBH in a low tumorigenic human breast carcinoma cell line increased the tumorigenicity of these breast cancer cells both in vitro and in vivo in a Xenograft mouse model. In addition, ectopic expression of LBH in normal adult mouse mammary epithelial cells resulted in increased self-renewal, inhibition of terminal cell differentiation, and most strikingly, repression of ER. This finding suggests that LBH overexpression in breast tumors may lead to ER-negativity, which is characteristic of metastatic, treatment-refractory breast cancers. Thus, LBH appears to control the self-renewal, maintenance and survival of normal and neoplastic breast epithelial stem cells. LBH is therefore a novel molecular marker for triple-negative breast cancers (those in which cancer cells do not express the genes for estrogen receptor, progesterone receptor, or Her2/neu) and is a novel target for elimination of malignant tumor-initiating cancer stem cells by killing these virulent cancer cells or by differentiation therapy. It was also discovered that WNT7A is an antagonist of canonical WNT signaling and LBH induction in triple-negative breast cancer. Thus, WNT7A and compositions including WNT7A may find use as inhibitors of cancer cell growth (e.g., tumor suppressors). Although many of the experiments described herein pertain to breast cancer, the compositions and methods described herein can be used for the detection and/or treatment of any type of cancer. For instance, it was discovered that LBH is also aberrantly overexpressed in human colon cancer correlating with hyperactivation of the WNT signaling pathway.

Accordingly, described herein is a composition including a therapeutically effective amount of an LBH inhibitor (e.g., LBH-specific siRNA, shRNA) for inhibiting cancer cell growth in a subject having cancer cells and a pharmaceutically acceptable carrier. The cancer cells can be breast cancer cells, triple-negative breast cancer cells.

Also described herein is a composition including a therapeutically effective amount of WNT7a protein or nucleic acids encoding WNT7a protein for inhibiting cancer cell growth in a subject having cancer cells and a pharmaceutically acceptable carrier. The cancer cells can be breast cancer cells, triple-negative breast cancer cells.

Further described herein is a method of inhibiting growth of cancer cells. The method includes contacting the cancer cells with a composition including a therapeutically effective amount for inhibiting cancer cell growth of at least one of: an LBH inhibitor, a WNT7a protein, and a nucleic acid encoding WNT7a protein, under conditions such that the cancer cells die or differentiate. The cancer cells can be, for example, triple-negative breast cancer cells. In one embodiment, the composition includes an LBH inhibitor and a WNT7a protein or a nucleic acid encoding WNT7a protein.

A method of treating a subject having estrogen receptor negative basal-type breast cancer is also described. The method includes administering to the subject a composition including a pharmaceutical carrier and at least one of: an LBH inhibitor, a WNT7a protein, and a nucleic acid encoding WNT7a protein in an amount effective for inhibiting growth of estrogen receptor negative basal-type breast cancer cells in the subject. In one embodiment, the composition includes an LBH inhibitor and a WNT7a protein or a nucleic acid encoding WNT7a protein. In one embodiment, the composition includes an LBH inhibitor such as LBH-specific siRNA.

Yet further described is a method of detecting the presence of cancer in a subject. The method includes the steps of: obtaining a biological sample from the subject; contacting the sample with at least one reagent that detects the presence of LBH expression; measuring the level of LBH expression in the biological sample; and correlating overexpression of LBH in the sample with the presence of cancer cells in the subject. In one embodiment, the cancer to be detected is estrogen receptor negative basal-type breast cancer. The at least one reagent can be, for example, an LBH-specific antibody. In another embodiment, the at least one reagent is one or more (e.g., two, three, four, five, etc.) LBH-specific primers for a polymerase chain reaction (PCR) analysis (e.g., real-time PCR).

Also described herein is a kit for detecting the presence of estrogen receptor-negative basal-type breast cancer in a subject. The kit includes at least one reagent (e.g., an LBH-specific antibody, a pair of LBH-specific primers for PCR, etc.) for detecting the presence of LBH expression and quantifying the expression of LBH in a biological sample from the subject; and instructions for use.

Unless otherwise defined, all technical terms used herein have the same meaning as commonly understood by one of ordinary skill in the art to which this invention belongs.

As used herein, “protein” and “polypeptide” are used synonymously to mean any peptide-linked chain of amino acids, regardless of length or post-translational modification, e.g., glycosylation or phosphorylation.

By the terms “LBH protein” or “LBH polypeptide” is meant an expression product of an LBH gene such as the native human LBH protein (SEQ ID NO:1; NM_{—}030915); accession no. NP_{—}112177), or a protein that shares at least 65% (but preferably 75, 80, 85, 90, 95, 96, 97, 98, or 99%) amino acid sequence identity with the foregoing and displays a functional activity of a native LBH protein. A “functional activity” of a protein is any activity associated with the physiological function of the protein. For example, functional activities of a native LBH protein may include transcriptional regulation of gene expression during embryonic development and lineage-specific progenitor cell proliferation and differentiation. LBH demonstrates selective expression in certain neoplastic tissues (e.g., estrogen receptor α (ER)-negative breast cancer cells that are characterized by an invasive basal-like and poorly differentiated phenotype).

As used herein, the phrases “LBH overexpression” and “overexpression of LBH” are used interchangeably to mean increased levels of LBH mRNA and protein expression as compared to normal tissues.

By the terms “WNT7A protein” or “WNT7A polypeptide” is meant an expression product of a WNT7A gene such as the native human WNT7A protein (SEQ ID NO:2; NM_{—}004625); accession no. NP_{—}004616) or a protein that shares at least 65% (but preferably 75, 80, 85, 90, 95, 96, 97, 98, or 99%) amino acid sequence identity with the foregoing and displays a functional activity of a native WNT7A protein. A “functional activity” of a protein is any activity associated with the physiological function of the protein. For example, functional activities of a native WNT7A protein may include activation of a poorly understood intracellular signal transduction pathway involving the homeodomain transcription factor LMX1B, regulation of dorsal/ventral limb patterning and other developmental processes during embryogenesis, and possible tumor suppressor in lung cancer.

By the term “gene” is meant a nucleic acid molecule that codes for a particular protein, or in certain cases, a functional or structural RNA molecule.

By the terms “LBH gene,” “LBH polynucleotide,” or “LBH nucleic acid” is meant a native human LBH-encoding nucleic acid sequence, e.g., the native human LBH gene (SEQ ID NO:3; accession no. NM_{—}030915); a nucleic acid having sequences from which a LBH cDNA can be transcribed; and/or allelic variants and homologs of the foregoing. The terms encompass double-stranded DNA, single-stranded DNA, and RNA.

By the terms “WNT7A gene,” “WNT7A polynucleotide,” or “WNT7A nucleic acid” is meant a native human WNT7A-encoding nucleic acid sequence, e.g., the native human WNT7A gene (SEQ ID NO:4; accession no. NM_{—}004625); a nucleic acid having sequences from which a WNT7A cDNA can be transcribed; and/or allelic variants and homologs of the foregoing. The terms encompass double-stranded DNA, single-stranded DNA, and RNA.

As used herein, a “nucleic acid” or a “nucleic acid molecule” means a chain of two or more nucleotides such as RNA (ribonucleic acid) and DNA (deoxyribonucleic acid).

The terms “patient,” “subject” and “individual” are used interchangeably herein, and mean a mammalian (e.g., human) subject to be treated and/or to obtain a biological sample from.

As used herein, “bind,” “binds,” or “interacts with” means that one molecule recognizes and adheres to a particular second molecule in a sample or organism, but does not substantially recognize or adhere to other structurally unrelated molecules in the sample. Generally, a first molecule that “specifically binds” a second molecule has a binding affinity greater than about 10⁸ to 10¹² moles/liter for that second molecule and involves precise “hand-in-a-glove” docking interactions that can be covalent and noncovalent (hydrogen bonding, hydrophobic, ionic, and van der waals).

The term “labeled,” with regard to a probe or antibody, is intended to encompass direct labeling of the probe or antibody by coupling (i.e., physically linking) a detectable substance to the probe or antibody.

When referring to a nucleic acid molecule or polypeptide, the term “native” refers to a naturally-occurring (e.g., a WT) nucleic acid or polypeptide.

As used herein, the terms “diagnostic,” “diagnose” and “diagnosed” mean identifying the presence or nature of a pathologic condition.

The term “sample” is used herein in its broadest sense. A sample including polynucleotides, peptides, antibodies and the like may include a bodily fluid, a soluble fraction of a cell preparation or media in which cells were grown, genomic DNA, RNA or cDNA, a cell, a tissue, skin, hair and the like. Examples of samples include saliva, serum, blood and plasma.

As used herein, the term “treatment” is defined as the application or administration of a therapeutic agent to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

By the term “differentiation therapy” is meant elimination of malignant cancer stem cells by treatments that induce these cells to terminally differentiate. During cell differentiation, these tumor cells lose their self-renewal capacity and can no longer seed new tumors.

As used herein, “sequence identity” means the percentage of identical subunits at corresponding positions in two sequences when the two sequences are aligned to maximize subunit matching, i.e., taking into account gaps and insertions. Sequence identity is present when a subunit position in both of the two sequences is occupied by the same nucleotide or amino acid, e.g., if a given position is occupied by an adenine in each of two DNA molecules, then the molecules are identical at that position. For example, if 7 positions in a sequence 10 nucleotides in length are identical to the corresponding positions in a second 10-nucleotide sequence, then the two sequences have 70% sequence identity. Sequence identity is typically measured using sequence analysis software (e.g., Sequence Analysis Software Package of the Genetics Computer Group, University of Wisconsin Biotechnology Center, 1710 University Avenue, Madison, Wis. 53705).

When referring to mutations in a nucleic acid molecule, “silent” changes are those that substitute one or more base pairs in the nucleotide sequence, but do not change the amino acid sequence of the polypeptide encoded by the sequence. “Conservative” changes are those in which at least one codon in the protein-coding region of the nucleic acid has been changed such that at least one amino acid of the polypeptide encoded by the nucleic acid sequence is substituted with another amino acid having similar characteristics.

As used herein, the terms “oligonucleotide”, “siRNA” “siRNA oligonucleotide” and “siRNA's” are used interchangeably throughout the specification and include linear or circular oligomers of natural and/or modified monomers or linkages, including deoxyribonucleosides, ribonucleosides, substituted and alpha-anomeric forms thereof, peptide nucleic acids (PNA), ed nucleic acids (LNA), phosphorothioate, methylphosphonate, and the like. Oligonucleotides are capable of specifically binding to a target polynucleotide by way of a regular pattern of monomer-to-monomer interactions, such as Watson-Crick type of base pairing, Hoogsteen or reverse Hoogsteen types of base pairing, or the like.

As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a composition of the present invention effective to yield the desired therapeutic response. For example, an amount effective to delay the growth of or to cause a cancer (breast cancer) to shrink or prevent metastasis. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

Although compositions, kits, transgenic animals and methods similar or equivalent to those described herein can be used in the practice or testing of the present invention, suitable compositions, kits, transgenic animals and methods are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

FIG. 1 is a schematic of the LBH protein, an alignment of consensus sequences, a pair of photographs of gel shifts, a schematic showing luciferase reporter constructs, and a graph showing identification of functional TCF-binding elements (TBE) in the mouse Lbh gene locus. (A) Schematic of genomic Lbh promoter/enhancer sequences in the 5′ upstream region and the first intron between exons 1 and 2 (black boxes). The positions of four putative conserved TBE sites (T1-T4; grey boxes) predicted by MatInspector (Genomatix) and/or rVista (WWW) computer software are shown in relationship to the transcriptional start site (+1). Sequences of T1-T4 in comparison with a TBE consensus site (van de Wetering et al., 1991) are indicated. S=sense strand; AS=antisense strand. The nucleic acid sequences from top to bottom are SEQ ID NOs:5-9, respectively. (B) Electrophoretic mobiliy shift assay (EMSA, also referred to herein as “gel shifts”) showing high affinity binding of recombinant His-tagged TCF4 protein to T1-T4. In oligonucleotide competition experiments, no competitor oligonucleotide (−), a 400-fold excess of unlabeled Wild-type TBE oligonucleotide (+), or mutant TBE oligonucleotide (m) was added to the gel shift reactions. The migration of free probe (brackets), specific TCF4 protein-DNA complexes (solid arrow) and unspecific complexes (arrowhead) are indicated. (C) Supershift of His-TCF4 binding to the T3 (+1558) site. Addition of 1-5 μg of anti-Histidine antibody (α-His) preferentially shifts the lower migrating molecular complex (arrow). (D) The functionality of the TCF-binding elements (T1-T4) in the Lbh gene locus was analyzed using transient reporter assays in HC11 mouse mammary epithelial cells. Luciferase (Luc) reporter constructs containing different murine Lbh promoter/enhancer sequences (P, E1, E2) with wild-type (wt) and mutant TBEs (T1-T4) as shown schematically were cotransfected with pCDNA3 constructs expressing constitutively active forms of either β-catenin (β-catenin^{S37Y}) or TCF4 (TCF4-VP16) respectively. Relative fold increase of transcriptional activation was calculated for each construct and plotted for the β-catenin cotransfection experiments. The values of the TCF4-VP16 cotransfection experiment are shown numerically plus/minus sd (standard deviation). *P<0.02.

FIG. 2 is a series of graphs showing endogenous LBH mRNA expression is induced by canonical Wnt signaling in human 293T kidney epithelial cells. (A)-(C) qPCR analysis measuring relative mRNA levels of LBH (red), DKK1 (blue), and β-catenin (yellow) normalized to mRNA levels of GAPDH in untreated as well as Wnt3a-treated 293T cells. All measurements were performed in triplicate. (A) Time course of induction of LBH and DKK1 expression in response to Wnt3a. Induction of both genes was inhibited by co-administration of recombinant Wnt inhibitor DKK1 8 hours after Wnt3a addition. (B) siRNA knockdown of β-catenin for 72 hours abolishes Wnt3a induced upregulation of LBH, confirming activation of LBH expression by the canonical Wnt signaling pathway. Note the reduction of β-catenin mRNA levels to less than 20% of endogenous expression in β-catenin (β-cat) siRNA transfected cells. (C) Treatment of cells with recombinant Wnt5a or Wnt7a blocked basal expression of LBH and DKK1. Wnt7a, but not Wnt5a, efficiently inhibited induction of LBH by Wnt3a similar to co-administration of Wnt antagonist DKK1.

FIG. 3 shows expression of Lbh during normal mouse mammary gland development and overexpression in Wnt-induced mammary tumors. (A) RNA in situ hybridization analysis of sagital cryosections of 7 week-virgin, 13 day-pregnant, 12 day-lactating and 4 day-involuting normal mammary glands (original magnification ×40). Lbh is expressed in basal-myoepithelial, terminal end bud and stromal cells in virgin mammary glands, as well as in the lobuloalveolar units during pregnancy and involution. Note, Lbh is not expressed in luminal epithelial cells or in lactating mammary glands. (B) Western blot analysis depicting Lbh protein levels during normal mammary gland development at the same stages as in (A). (C) RNA in situ hybridization, and (D) Western Blot analysis showing elevated Lbh expression levels in mammary tumors of MMTV-Wnt1 transgenic mice (T1-T6) as compared to HC11 and isolated wild-type (WT) mammary epithelial cells (MEC). Basal (Keratin 5) and luminal (Keratin 8) mammary epithelial markers, as well as an β-actin loading control are shown. Quantification of Lbh protein levels by densitometry normalized to β-actin values is shown in bottom panel.

FIG. 4 shows validation of LBH expression in human breast tumor cell lines. (A) LBH mRNA expression is significantly higher in basal rather than luminal breast carcinoma cell lines classified according to tumor subtype (Neve et al., Cancer Cell 10:515-27, 2006). Values represent the mean and error bars the standard error. (n)=number of samples per tumor subtype. (B) qPCR analysis of relative LBH mRNA expression in a panel of human breast tumor cell lines showing overexpression of LBH in HCC1395, MDA-MB-231 and HCC1187 tumor cells. Cell lines are arranged by tumor subtype (Neve et al., Cancer Cell 10:515-27, 2006). All measurements were performed in triplicate and expression levels were normalized to mRNA levels of GAPDH. (C) Comparative genomic hybridization array (aCGH) analysis of the same breast tumor cell lines as in (B). (D) Western blot analysis detecting expression of LBH protein exclusively in invasive ER-negative basal-type breast cancer lines, but not in two non-transformed (normal) mammary epithelial cell lines or in low-invasive breast tumor cell lines. β-actin was used as a loading control. (E) TOPFlash reporter assay detects Wnt signaling activity in LBH-expressing HCC1395 and HCC1187 cells, but not in MDA-MB-231 cells. HC11 and HC11 transiently transfected with pcDNA3/β-catenin^{S37Y} were used as negative and positive controls, respectively. Values represent the mean ratio of TOPFlash over FOPFlash activity ±SD (F) Administration of recombinant DKK1 and Wnt7a (100 μg/ml) for the indicated time points strongly inhibits LBH and DKK1 mRNA expression in HCC1395 cells as revealed by qPCR analysis. Values represent mean±SEM (n=3).

FIG. 5 is a plot, a graph, a scatter plot, and a Kaplan-Meier curve showing LBH gene expression in human breast tumors correlates with basal-like tumor subtype and poor clinical outcome. (A) Meta-analysis of 1107 human primary breast carcinoma samples from six published Affimetrix datasets (Sims et al., 2008) showing a strong correlation of LBH expression with basal tumor type as well as with expression of the Wnt signaling components TCF4 and TCF7. In contrast, the LBH signature inversely correlates with Estrogen Receptor α (ESR1) expression. Clustering of tumor subtypes: basal (red), ERBB2 (purple), luminal A (dark blue), luminal B (light blue) and normal-like (green) was according to (Sorlie et al., 2003). Red=high expression, green=low expression. (B) Graphical representation of the percentage of primary breast tumors with high LBH expression in each tumor subtype. (C) Scatter Plot showing inverse correlation of LBH with Estrogen receptor α (ESR1) expression in the data sets analyzed (R=−0.15, p<0.0001). (D) Kaplan-Meier curves for the combined ER negative (ER−) breast tumor cohorts (239 tumors) from five datasets depicting metastasis-free survival of patients whose primary tumors expressed greater than median levels of LBH (LB^{Hhigh}, green) and those that expressed less-than-median levels of LBH (LB^{Hlow}, blue). P-value is shown. ER status was determined by immunohistochemistry.

FIG. 6 is a series of Dot plots demonstrating correlations between LBH expression and clinical markers (Top panel) and Wnt pathway genes (Lower panel). Pearson correlation coefficients (R values) between LBH expression and other genes are listed. LBH inversely correlates with estrogen receptor (ESR1) gene expression, has no correlation with ERBB2, and positively correlates with expression of basal Keratin 5 (KRT5). LBH expression also positively correlates with expression of Wnt pathway genes that are also targets of canonical Wnt signaling, including Secreted Frizzled-Related Protein 1 (SFRP1), TCF7, TCF4, and Dickkopf 3 (DKK3). All R values are statistically significant (p<0.0001) with the exception of ERBB2.

FIG. 7 is a pair of FACS analysis plots from a FACS analysis that reveals a decrease in the percentage of CD44+/CD24− tumor cells in the HCC1395 breast carcinoma cell line after siRNA mediated knockdown of LBH for a period of nine days. Red boxes indicate the population of CD44^{high}/CD24^{low} cells. Yellow boxes indicate the population of CD44^{high}/CD24^{high} cells.

FIG. 8 shows Wnt/β-catenin-mediated induction and ectopic expression of Lbh in HC11 mammary epithelial cells. (A) Immunofluorescence analysis showing nuclear translocation of β-catenin in HC11 cells treated with Wnt3a conditioned media (+Wnt3a) for 6 h, but not in untreated cells. (B) qPCR analysis measuring rapid induction of Lbh in cells treated with Wnt3a at the indicated time points. (C) ChIP analysis of β-catenin occupancy of endogenous Lbh gene regulatory sequences (T1/2, T4) in cells treated with Wnt3a for 3 h. DNA derived from sheared chromatin fragments from untreated and Wnt3a-treated cells immunoprecipitated with antibodies to β-catenin, acetyl Histone 3 and normal rabbit IgG was quantified by semi-quantitative RT-PCR. As a control, <1% of input chromatin was used. (D) qPCR (Top) and Western blot analyses (bottom) of two polyclonal cultures (c1 and c2) of HC11 cells stably expressing pCDNA3-NLbh or pCDNA3 vector alone, showing overexpression of Lbh in HC11-Lbh cultures. β-actin was used as a loading control. (E) Ectopic expression of Lbh increases cell viability as assessed by CellTiter 96® AQueous One solution cell proliferation assay. Values represent the mean value; error bars represent the SD (n=3). Students t-test was used to evaluate significance: *P<0.01, **P<0.001. (F) Semi-quantitative RT-PCR (Top) and qPCR (Bottom) analyses measuring induction of the terminal differentiation marker β-casein. Confluent cell cultures were treated for 3 days with normal growth media or serum-free differentiation media containing 5 μg/ml Prolactin and 1 μM Dexamathasone (PRL/DEX). qPCR values were normalized to Gapdh. All values represent mean±SEM (n=3).

FIG. 9 is a schematic and a pair of photographs of Southern blots showing construction of a conditionally mutant LBH allele. (A) Gene targeting scheme for conditional Lbh gene activation based on the Cre-loxP system. (B) Southern Blots of ES cell DNA digested with EcoRI and NcoI hybridized with genomic fragments external (P1) and internal (P2) to the targeting vector. A genomic targeting event is apparent by size change of one allele in three ES cell clones.

FIG. 10 is a pair of micrographs showing IHC anti-LBH staining of paraffin-embedded sections of normal human breast and a triple-negative metaplastic breast tumor. Note the expression of LBH in basal mammary epithelial cells in normal breast and its overexpression in triple-negative metaplastic tumor cells.

FIG. 11 is a series of graphs, plots and photographs of electrophoretic gels showing that RNAi knockdown (KD) of LBH in TNBC breast tumor cells lines reduces the CD44^{high}/CD24^{low} CSC population through differentiation and apoptosis induction. (A) qPCR showing efficient LBH KD in basal tumor lines 6 or 9 days (d) after transfection with LBH-specific siRNAs (Darmacon). Bars represent the mean of 3 replicates ±SD. (B) Western Blot demonstrating long-lasting (>9 d) LBH KD in transfected HCC1395 cells. (C) FACS analysis showing a 25% reduction in the CD44^{high}/CD24^{low} TIC population and a reciprocal increase in the more differentiated CD44^{high}/CD24^{high} tumor cell population upon LBH KD for 9 d. A representative experiment (n=3) is shown. (D) qPCR and (E) Western Blot analysis detecting upregulation of luminal marker CD24 in LBH KD tumor cells. (F) MTT assay (Promega) showing reduced growth of LBH-KD HCC1395 cells. *P<0.005. Cells were seeded 3 d after siRNA transfection and grown in growth medium for the indicated time points (G) Reduced colony formation of LBH-KD cells in soft agar. (H) FACS analysis for apoptosis marker Annexin V shows increased apoptosis of HCC1395 cells upon 6 d of LBH KD. (I) Lentivirally-transduced stable KD of LBH results in ˜80% reduction of LBH mRNA and protein levels with shRNA clone 1 and 5 as revealed by qPCR (top panel) and WB (bottom panel) analyses. (J) Bright field images (20×) of HCC1395 stably transduced with control scrambled shRNA and LBH-specific shRNA lentivirus clone 1 after 2 weeks of puromycin (5 μg/ml) selection showing pronounced cell death of LBH-KD cells. (K-L) Ectopic expression of LBH increases tumorigenicity of BT549 basal breast carcinoma cells. (K) WB analysis of polyclonal cell cultures (c1-3) stably transfected with either pCDNA3 vector or a pCDNA3-LBH expression plasmid using nucleofection (Amaxa) followed by selection in G418 (350 μg/ml). (L) Increased colony formation of BT549+LBH cells in soft agar. Bright field images (20×) and statistical evaluation of a representative experiment (n=3) are shown. (Bottom panel) Quantification of colonies >8 pixies at 100% magnification in Photoshop of three 35 mm dishes per cell line were counted. Bars represent the mean±SD. P<0.0001.

FIG. 12 is plot of results from a Meta-analysis of 281 colon tumors from the Expo data set that was performed and that demonstrates that LBH (denoted by the arrow) is expressed in a subset of colon tumors. Like in breast cancer, LBH expression positively correlates with expression of a subset of Wnt target genes including TCF4, SFRP1, and DKK3. The Pearson correlation coefficients are shown to the right.

### DETAILED DESCRIPTION

Described herein are compositions, methods and kits for detecting and treating cancer (e.g., breast cancer), as well as transgenic animals for analyzing LBH gene function and molecular markers that control stem cell biology. The experiments described herein show that LBH is a direct transcriptional target of the canonical WNT/β-catenin signaling pathway and that LBH is expressed at abnormally high levels in mammary tumors of MMTV-Wnt1 transgenic mice, further underscoring the biological significance of LBH as a WNT target gene. The experimental results show that LBH is deregulated, alongside other WNT pathway genes, in human basal-like breast carcinomas, a form of breast cancer that is characterized by a highly invasive, poorly differentiated tumor phenotype with poor clinical outcome. The data described herein provide the first evidence that LBH may function as a downstream effector of WNT/β-catenin signaling in embryonic development, as well as in WNT-induced oncogenesis, and that WNT7A is an antagonist of canonical WNT signaling and LBH induction in triple-negative breast cancer.

### Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Conventional methods of gene transfer and gene therapy can also be adapted for use in the present invention. See, e.g., Gene Therapy: Principles and Applications, ed. T. Blackenstein, Springer Verlag, 1999; Gene Therapy Protocols (Methods in Molecular Medicine), ed. P. D. Robbins, Humana Press, 1997; and Retro-vectors for Human Gene Therapy, ed. C. P. Hodgson, Springer Verlag, 1996.

### LBH Proteins

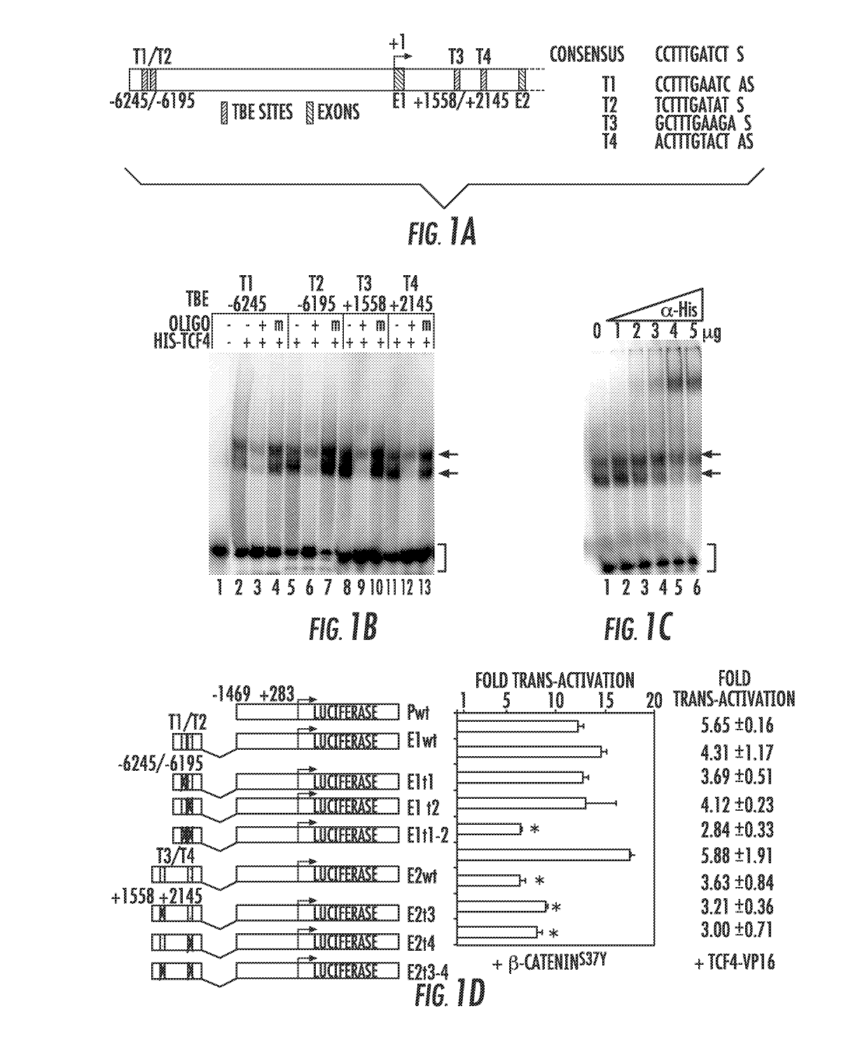

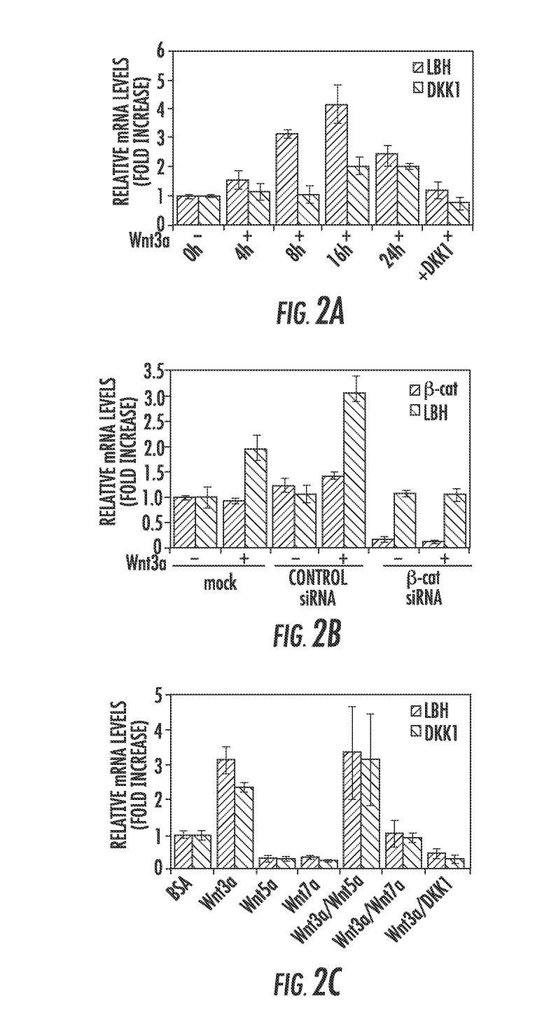

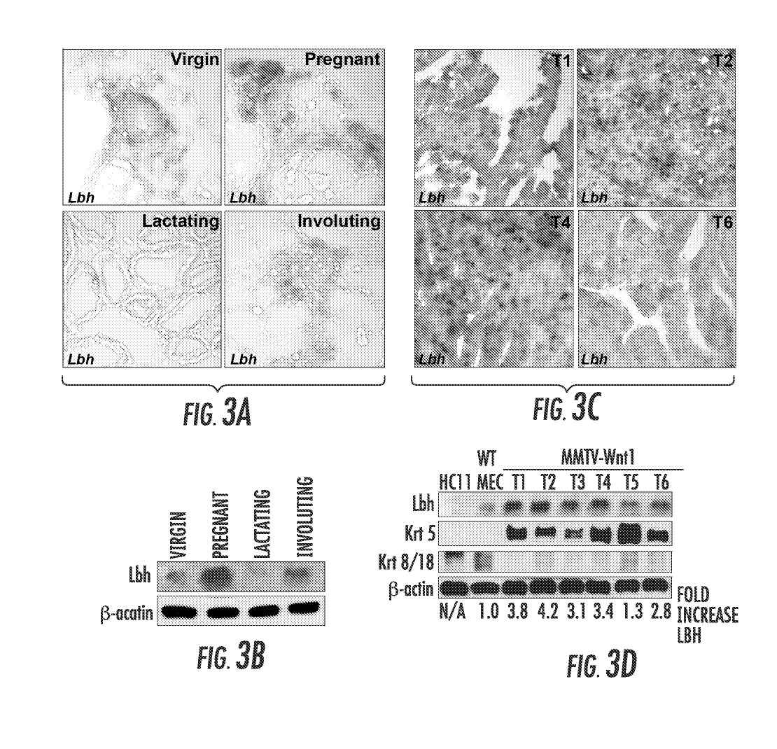

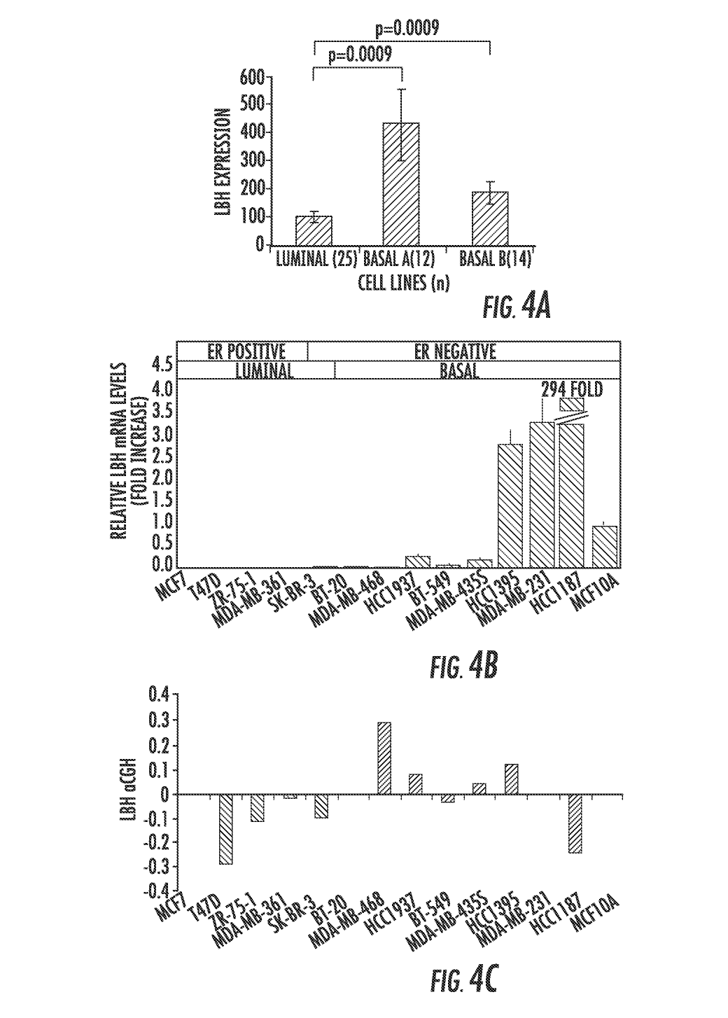

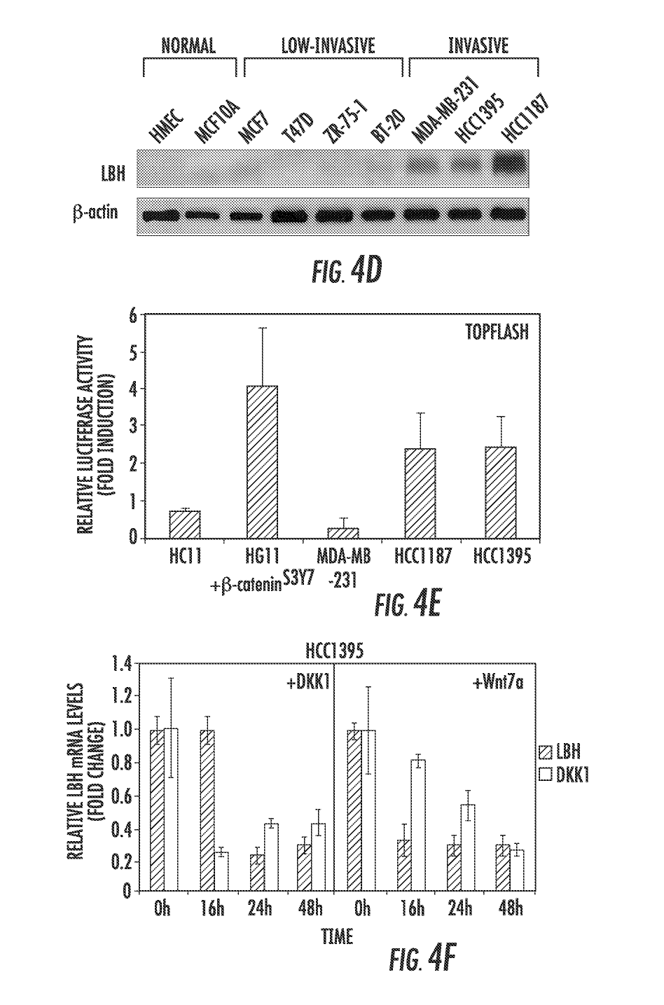

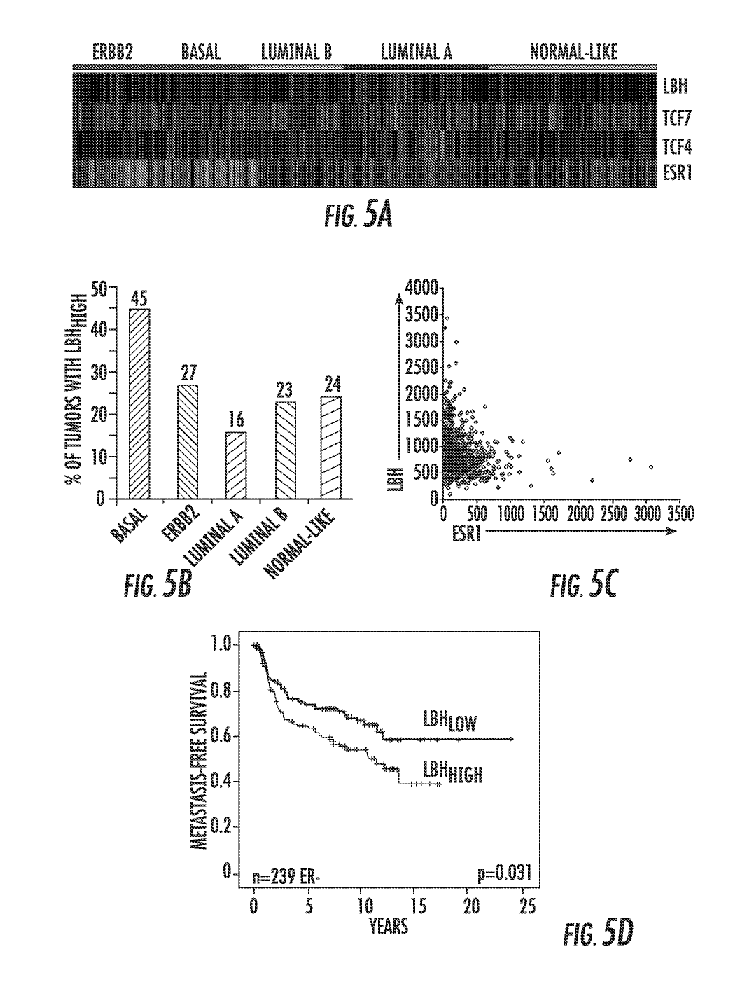

Described herein are methods and kits for detecting LBH protein in a sample from a subject to detect the presence of cancer (e.g., breast cancer) in the subject, compositions and methods for modulating LBH expression and activity, a conditional LBH mouse model, as well as compositions, kits and methods for inhibiting LBH expression or activity to treat a subject having cancer (e.g., breast cancer). LBH encodes a unique, highly conserved vertebrate protein of 105 amino acids (12.3 kDa) with no structural homology to any other known protein family. A Xenopus orthologue of Lbh, termed XlCl2 (=Xenopus laevis Cleavage RNA 2), was cloned as a maternal factor of unknown function that gets activated by polyadenylation upon fertilization. The mouse, human and bovine LBH proteins share 90% amino acid identity with each other, while mammalian LBH proteins share 77%-80% amino acid identity with the frog XlCl2 protein (Briegel and Joyner, Dev Biol 233, 291-304, 2001). There are no LBH homologues in invertebrates or lower species, indicating that LBH originated in metazoa during the course of evolution of higher ordered structures. LBH/XlCl2 proteins possess a nuclear localization signal (NLS) and an acidic Glutamate-rich putative transcriptional activation (TA) domain at their C-terminus, but no DNA-binding domain (FIG. 1A; Briegel and Joyner, Dev Biol 233, 291-304, 2001). Moreover, LBH has several putative phosphorylation sites, suggesting that LBH protein activity might be regulated by different mitogenic signaling pathways. In keeping with this primary protein structure, LBH localizes to the nucleus and can both activate and repress transcription in cell-based reporter assays depending on the transcription factor context (FIG. 2; Briegel, Development 132, 3305-3316). These data indicate that LBH acts as a tissue-specific transcription cofactor. The unique spatio-temporal expression pattern of LBH during vertebrate embryogenesis suggests that LBH may act downstream of morphogenic signaling pathways and play important roles in lineage-specific progenitor cell specification, proliferation, and differentiation (Briegel and Joyner, Dev Biol 233, 291-304, 2001).

### LBH Inhibitor Compositions for Inhibiting Cancer Cell Growth

Compositions described herein for inhibiting cancer cell growth include a therapeutically effective amount of an inhibitor of LBH for inhibiting cancer (e.g., estrogen receptor negative basal-type breast cancer) cell growth and a pharmaceutically acceptable carrier. Any suitable inhibitor of LBH activity or expression can be used. Such compositions can be used to inhibit growth of any type of cancer cell that overexpresses LBH, such as estrogen-receptor negative breast cancer, colon cancer, lung cancer and others (e.g. skin, hematopoietic cancers). In addition to breast cancer, examples of cancers that can be inhibited using the compositions include colon cancer, lung cancer and others (e.g. skin).

An inhibitor of LBH reduces the level of LBH in a cell and/or reduces the activity of LBH in a cell. Any agent that reduces the level of LBH in a cell and/or reduces the activity of LBH in a cell can be used. An inhibitor of LBH active to reduce the level of LBH protein in the cell may be an inhibitor of transcription and/or translation of LBH. In addition, an inhibitor of LBH active to reduce the level of LBH protein in the cell may stimulate degradation of the LBH protein and/or LBH encoding RNA. An inhibitor of LBH transcription and/or translation may be a nucleic acid-based inhibitor such as an antisense oligonucleotides complementary to a target LBH mRNA, as well as ribozymes and DNA enzymes which are catalytically active to cleave the target mRNA. Examples of additional LBH inhibitors include WNT7a, which blocks LBH at the transcriptional level, or inhibitors of kinases/phosphatases that prevent phosphorylation of LBH, which may control LBH activity and localization in the cell. Small molecule inhibitors that inhibit LBH activity by altering its protein conformation or by interfering with essential protein-protein interactions. Inhibiting cancer cell growth includes inducing death (killing of) of the cancer cells, and/or inducing differentiation of the cancer cells (promoting a more differentiated phenotype).

In some embodiments, an inhibitor of LBH, when administered to a subject having cancer stem cells, reduces the ability of the cancer stem cells to maintain their stem cell characteristics and/or induces cancer cell death. In the experiments described herein, LBH was depleted in triple-negative breast cancer cell lines via RNAi knockdown, and the results showed that LBH depletion leads to acquisition of a more luminal tumor phenotype (a more differentiated phenotype), which has a better prognosis. In other words, the depletion of LBH in these cell populations resulted in a significant decrease in the number of cells exhibiting stem cell characteristics. Also in the experiments described herein, LBH depletion resulted in reduced cell viability and reduced anchorage-independent growth. More permanent depletion of LBH in triple-negative breast cancer cell lines that mainly consist of CSCs (83-90%) resulted in almost complete tumor cell death, suggesting that inhibition of LBH efficiently kills CSCs.

In a typical embodiment, a composition described herein includes an LBH-specific siRNA. Sequence specific siRNA bind to a target nucleic acid molecule, inhibiting the expression thereof. siRNA's are effective in the treatment of abnormal cells, abnormal cell growth and tumors, including those tumors caused by infectious disease agents. Compositions for delivery of siRNA and methods of treatment thereof are provided. In the experiments described herein, LBH-specific siRNAs were used to knock-down (deplete) expression of LBH.

Methods of constructing and using ribozymes, siRNA and antisense molecules are known in the art (e.g., Isaka Y., Curr Opin Mol Ther vol. 9:132-136, 2007; Sioud M. and Iversen P. O., Curr Drug Targets vol. 6:647-653, 2005; Ribozymes and siRNA Protocols (Methods in Molecular Biology) by Mouldy Sioud, ^{2nd} ed., 2004, Humana Press, New York, N.Y.). An “antisense” nucleic acid can include a nucleotide sequence which is complementary to a “sense” nucleic acid encoding a protein, e.g., complementary to the coding strand of a double-stranded cDNA molecule or complementary to an mRNA sequence. The antisense nucleic acid can be complementary to an entire LBH coding strand, or to only a portion thereof. In another embodiment, the antisense nucleic acid molecule is antisense to a “noncoding region” of the coding strand of a nucleotide sequence encoding LBH (e.g., the 5′ and 3′ untranslated regions). Anti-sense agents can include, for example, from about 8 to about 80 nucleobases (i.e. from about 8 to about 80 nucleotides), e.g., about 8 to about 50 nucleobases, or about 12 to about 30 nucleobases. Antisense compounds include ribozymes, external guide sequence (EGS) oligonucleotides (oligozymes), and other short catalytic RNAs or catalytic oligonucleotides which hybridize to the target nucleic acid and modulate its expression. Anti-sense compounds can include a stretch of at least eight consecutive nucleobases that are complementary to a sequence in the target gene. An oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

### WNT7A Compositions for Inhibiting Cancer Cell Growth

In other embodiments, compositions described herein for inhibiting cancer cell growth include WNT7A proteins or nucleic acids that encode WNT7A proteins for inhibiting cancer cell growth (e.g., estrogen receptor negative basal-type breast cancer) and a pharmaceutically acceptable carrier. As with the compositions including an inhibitor of LBH for inhibiting cancer cell growth described above, compositions that include Wnt7a proteins or nucleic acids that encode WNT7A proteins can be used to inhibit growth of any type of cancer cell that exhibits hyperactivation of the canonical WNT signaling pathway and/or overexpresses LBH. In addition to breast cancer, examples of cancers that can be inhibited using the compositions include colon cancer, lung cancer, endometrial cancer, ovarian cancer, etc.

A typical nucleic acid that encodes WNT7A is the native human WNT7A nucleic acid deposited with Genbank as accession no. NM_{—}004625. Nucleic acid molecules as described herein may be in the form of RNA or in the form of DNA (e.g., cDNA, genomic DNA, and synthetic DNA). The DNA may be double-stranded or single-stranded, and if single-stranded may be the coding (sense) strand or non-coding (anti-sense) strand. The coding sequence which encodes a native WNT7A protein may be identical to the nucleotide sequence of SEQ ID NO:4 (accession no. NM_{—}004625) or it may also be a different coding sequence which, as a result of the redundancy or degeneracy of the genetic code, encodes the same polypeptide as the polynucleotide of SEQ ID NO:2 (accession no. NP_{—}004616). Other nucleic acid molecules as described herein include variants of the native Wnt7a gene such as those that encode fragments, analogs and derivatives of a native WNT7A protein. Such variants may be, e.g., a naturally occurring allelic variant of the native WNT7A gene, a homolog of the native WNT7A gene, or a non-naturally occurring variant of the native WNT7A gene. These variants have a nucleotide sequence that differs from the native WNT7A gene in one or more bases. For example, the nucleotide sequence of such variants can feature a deletion, addition, or substitution of one or more nucleotides of the native WNT7A gene.

In other embodiments, variant WNT7A proteins displaying substantial changes in structure can be generated by making nucleotide substitutions that cause less than conservative changes in the encoded polypeptide. Examples of such nucleotide substitutions are those that cause changes in (a) the structure of the polypeptide backbone; (b) the charge or hydrophobicity of the polypeptide; or (c) the bulk of an amino acid side chain. Nucleotide substitutions generally expected to produce the greatest changes in protein properties are those that cause non-conservative changes in codons. Examples of codon changes that are likely to cause major changes in protein structure are those that cause substitution of (a) a hydrophilic residue, e.g., serine or threonine, for (or by) a hydrophobic residue, e.g., leucine, isoleucine, phenylalanine, valine or alanine; (b) a cysteine or proline for (or by) any other residue; (c) a residue having an electropositive side chain, e.g., lysine, arginine, or histidine, for (or by) an electronegative residue, e.g., glutamine or aspartine; or (d) a residue having a bulky side chain, e.g., phenylalanine, for (or by) one not having a side chain, e.g., glycine.

Naturally occurring allelic variants of a native WNT7A gene or native WNT7A mRNAs as described herein are nucleic acids isolated from human tissue that have at least 75% (e.g., 76%, 77%, 78%, 79%, 80%, 81%, 82%, 83%, 84%, 85%, 86%, 87%, 88%, 89%, 90%, 92%, 93%, 94%, 95%, 96%, 97%, 98%, and 99%) sequence identity with the native WNT7A gene or native WNT7A mRNAs, and encode polypeptides having structural similarity to a native WNT7A protein. Homologs of the native WNT7A gene or native WNT7A mRNAs as described herein are nucleic acids isolated from other species that have at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity with the native human WNT7A gene or native human WNT7Aa mRNAs, and encode polypeptides having structural similarity to native human WNT7A protein. Public and/or proprietary nucleic acid databases can be searched to identify other nucleic acid molecules having a high percent (e.g., 70, 80, 90% or more) sequence identity to the native WNT7A gene or native WNT7A mRNAs:

Non-naturally occurring WNT7A gene or mRNA variants are nucleic acids that do not occur in nature (e.g., are made by the hand of man), have at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity with the native human WNT7A gene or native human WNT7A mRNAs, and encode polypeptides having structural similarity to native human WNT7A protein. Examples of non-naturally occurring WNT7A gene variants are those that encode a fragment of a Wnt7a protein, those that hybridize to the native WNT7A gene or a complement of the native WNT7A gene under stringent conditions, those that share at least 65% sequence identity with the native WNT7A gene or a complement thereof, and those that encode a Wnt7a fusion protein.

Nucleic acids encoding fragments of a native WNT7A protein as described herein are those that encode, e.g., 2, 5, 10, 25, 50, 100, 150, 200, 300 or more amino acid residues of the native WNT7A protein. Shorter oligonucleotides (e.g., those of 6, 7, 8, 9, 10, 11, 12, 13, 14, 15, 16, 17, 18, 19, 20, 30, 50, base pairs in length) that encode or hybridize with nucleic acids that encode fragments of a native WNT7A protein can be used as probes, primers, or antisense molecules. Nucleic acids encoding fragments of a native WNT7A protein can be made by enzymatic digestion (e.g., using a restriction enzyme) or chemical degradation of the full length native WNT7A gene, a WNT7A mRNA or cDNA, or variants of the foregoing. Using the nucleotide sequence of the native human WNT7A gene and the amino acid sequence of the native WNT7A protein previously reported, those skilled in the art can create nucleic acid molecules that have minor variations in their nucleotide sequence, by, for example, standard nucleic acid mutagenesis techniques or by chemical synthesis. Variant WNT7A nucleic acid molecules can be expressed to produce variant WNT7A proteins.

In some embodiments, a composition including WNT7A proteins or nucleic acids that encode Wnt7a proteins, when administered to a subject having cancer stem cells, reduces the ability of the cancer stem cells to maintain their stem cell characteristics. The most efficient form of using WNT7A would be to synthesize active WNT7A-specific peptides that efficiently bind to the WNT7A receptor.

### Effective Doses

The compositions described above are preferably administered to a mammal (e.g., rodent, human) in an effective amount, that is, an amount capable of producing a desirable result in a treated subject (e.g., inhibiting growth of breast cancer cells and metastasis of breast cancer cells in the subject). Toxicity and therapeutic efficacy of the compositions utilized in methods of the invention can be determined by standard pharmaceutical procedures. As is well known in the medical and veterinary arts, dosage for any one animal depends on many factors, including the subject's size, body surface area, age, the particular composition to be administered, time and route of administration, general health, and other drugs being administered concurrently.

The amount of the therapeutic agent to be administered varies depending upon the manner of administration, the age and body weight of the patient, and with the clinical symptoms of the cancer. A composition as described herein is typically administered at a dosage that inhibits LBH biological activity and/or expression, as assayed by identifying a reduction in tumor growth rate, tumor size, neovasculogenesis, or cancer cell growth or proliferation, or using any that assay that measures the expression or the biological activity of an LBH polypeptide.

### Methods of Treating Cancer

Described herein are methods of treating cancer (e.g., breast cancer) and/or disorders or symptoms thereof which include administering a therapeutically effective amount of a pharmaceutical composition including an LBH inhibitor, and/or a nucleic acid encoding WNT7A or WNT7A polypeptides, and small synthetic WNT7A-active peptides to a subject (e.g., a mammal such as a human). Thus, one embodiment is a method of treating a subject suffering from cancer (e.g., breast cancer), or disorder or symptom thereof. The method includes administering to the mammal a therapeutic amount of a composition including an LBH inhibitor, a nucleic acid encoding WNT7A, or WNT7A polypeptides, and small synthetic WNT7A-active peptides to a subject sufficient to treat the disease or disorder or symptom thereof.

The therapeutic methods of the invention (which include prophylactic treatment) in general include administration of a therapeutically effective amount of the compositions described herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, marker (as defined herein), family history, and the like). The compositions herein may be also used in the treatment of any other disorders in which an excess of WNT signaling, LBH expression, or activity may be implicated.

In one embodiment, the invention provides a method of monitoring treatment progress. The method includes the step of determining a level of diagnostic marker such as LBH (e.g., any target delineated herein modulated by a composition or agent described herein, a protein or indicator thereof, etc.) or diagnostic measurement (e.g., screen, assay) in a subject suffering from or susceptible to a disorder or symptoms thereof associated with cancer (e.g., breast cancer) in which the subject has been administered a therapeutic amount of a composition as described herein sufficient to treat the disease or symptoms thereof. The level of marker determined in the method can be compared to known levels of marker in either healthy normal controls or in other afflicted patients to establish the subject's disease status. In preferred embodiments, a second level of marker (e.g., LBH) in the subject is determined at a time point later than the determination of the first level, and the two levels are compared to monitor the course of disease or the efficacy of the therapy. In certain preferred embodiments, a pre-treatment level of marker in the subject is determined prior to beginning treatment according to the methods described herein; this pre-treatment level of marker can then be compared to the level of marker in the subject after the treatment commences, to determine the efficacy of the treatment.

The administration of composition including an LBH inhibitor, a nucleic acid encoding WNT7A, or WNT7A polypeptides, and small synthetic WNT7A-active peptides for the treatment of cancer (e.g., breast cancer) may be by any suitable means that results in a concentration of the therapeutic that, combined with other components, is effective in ameliorating, reducing, or stabilizing a neoplasia. The LBH inhibitor, a nucleic acid encoding WNT7A, or WNT7A polypeptides, and small synthetic WNT7A-active peptides may be contained in any appropriate amount in any suitable carrier substance, and is generally present in an amount of 1-95% by weight of the total weight of the composition. The composition may be provided in a dosage form that is suitable for local or systemic administration (e.g., intratumoral, parenteral, subcutaneously, intravenously, intramuscularly, or intraperitoneally). The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Compositions as described herein may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a neoplasia, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing agents.

As indicated above, the pharmaceutical compositions described herein may be in the form suitable for sterile injection. To prepare such a composition, the suitable active therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

Formulations for oral use include tablets containing the active ingredient(s) (e.g., LBH inhibitor and/or WNT7a) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

The solid tablet compositions may include a coating adapted to protect the composition from unwanted chemical changes, (e.g., chemical degradation prior to the release of the active anti-neoplasia therapeutic substance). The coating may be applied on the solid dosage form in a similar manner as that described in Encyclopedia of Pharmaceutical Technology, supra.

At least two anti-cancer therapeutics (e.g., an LBH inhibitor and a nucleic acid encoding WNT7A or WNT7A polypeptides and small synthetic WNT7A-active peptides) may be mixed together in the tablet, or may be partitioned. In one example, the first active anti-neoplasia therapeutic is contained on the inside of the tablet, and the second active anti-neoplasia therapeutic is on the outside, such that a substantial portion of the second active anti-neoplasia therapeutic is released prior to the release of the first active anti-neoplasia therapeutic.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment. Compositions as described herein can also be formulated for inhalation and topical applications.

Optionally, an anti-cancer therapeutic may be administered in combination with any other standard anti-cancer therapy; such methods are known to the skilled artisan and described in Remington's Pharmaceutical Sciences by E. W. Martin. In one example, an effective amount of an LBH inhibitor, a nucleic acid encoding WNT7a, or WNT7a polypeptides is administered in combination with radiation therapy. Combinations are expected to be advantageously synergistic. Therapeutic combinations that inhibit cancer (e.g., breast cancer) cell growth and/or induce apoptosis of cancer (e.g., breast cancer) cells are identified as useful in the methods described herein.

Further described herein are methods of detecting the presence of estrogen receptor negative basal-type breast cancer in a subject (e.g., human). The method includes obtaining a biological sample from the subject; contacting the sample with at least one reagent that detects presence of LBH expression; measuring the level of LBH expression in the biological sample; and correlating overexpression of LBH with the presence of estrogen receptor negative basal-type breast cancer in the subject. Any suitable reagent for detecting LBH expression can be used. In a typical embodiment, an LBH-specific antibody (e.g., monoclonal, polyclonal, Fab fragment, etc.) is used. In some embodiments, a method involving LBH-specific real-time PCR can be used to diagnose cancer in a subject. This method may be particularly useful for detecting circulating tumor cells in the blood of a subject.

### Kits

Described herein are kits for detecting the presence of cancer (e.g., estrogen receptor negative basal-type breast cancer) in a subject (e.g., human). A typical kit includes at least one reagent for detecting the presence of LBH expression in a biological sample from the subject and instructions for use. In one embodiment, a kit includes a monoclonal or polyclonal antibody to LBH, a detectable label, and instructions for use. In another embodiment, the kit includes LBH-specific primers for PCR, e.g., real-time PCR.

Kits for administering treatment to a subject (e.g., human) suffering from cancer (e.g., breast cancer) are also described herein. In one embodiment, the kit includes a therapeutic or prophylactic composition containing a therapeutically effective amount of an LBH inhibitor for inhibiting cancer (e.g., breast cancer) cell growth and a pharmaceutically acceptable carrier in unit dosage form. If desired, the kit also contains an effective amount of a nucleic acid encoding WNT7A or WNT7A polypeptides and small synthetic WNT7A-active peptides. In another embodiment, the kit includes a therapeutic or prophylactic composition containing an effective amount of a nucleic acid encoding WNT7A or WNT7A polypeptides and small synthetic WNT7A-active peptides in unit dosage form. Generally, a kit as described herein includes instructions for use. In some embodiments, the kit includes a sterile container which contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

### Conditional LBH Mouse Model

Described herein is a transgenic mouse wherein the Lbh gene is conditionally inactivated. The Lbh gene is conditionally inactivated because exon 2 of the Lbh gene is flanked with two loxP sites and cre recombinase-mediated recombination between the two loxP sites results in deletion of exon 2 and a frameshift mutation in the coding sequences of exon 3. The conditional LBH mouse model is useful for many applications, including analyzing LBH gene function, and deciphering the molecular mechanisms that control stem cell biology which is useful for regenerative medicine applications.

### EXAMPLES

The present invention is further illustrated by the following specific examples. The examples are provided for illustration only and should not be construed as limiting the scope of the invention in any way.

### Example 1

### Determination of the Stem Cell Content in Basal-Type Breast Cancer which Express LBH and Assaying Changes in Stem Cell Content Upon Modulation of LBH Expression

To date, several different populations of breast cancer stem cells have been identified. The first population of human breast cancer stem cells is characterized by high expression of the cell surface antigen CD44, and low expression of the antigen CD24 (CD44^{high}/CD24^{low}) (Al-Hajj et al., Proc Natl Acad Sci USA vol. 100:3983-3988, 2003). This cell population contains cells with increased metastatic potential (Fillmore and Kuperwasser Breast Cancer Res. vol. 10:R25, 2008,) and increased tumorigenicity in in vivo xenograft models (Al-Hajj et al., Proc Natl Acad Sci USA vol. 100:3983-3988, 2003). It has also been shown that these cells are resistant and become enriched during chemotherapy and radiation (Fillmore and Kuperwasser Breast Cancer Res. vol. 10:R25, 2008, Phillips et al., J Natl Cancer Inst vol. 98:1777-1785, 2006). A more recently identified population of breast cancer stem cells is characterized by high levels of Aldehyde dehydrogenase activity (ALDH+), which can also be detected using a fluorescent assay specific for measuring the activity levels of ALDH. This population of stem cells appears to show similar characteristics to the above mentioned population of stem cells (Croker et al., Journal of Cellular and Molecular Medicine vol. 9999, 2008; Tanei et al., Clin Cancer Res p. 1078-4032, CCR-08-1479, 2009). FACS analysis was performed on several LBH-positive human breast cancer cell lines to identify the proportion of breast cancer stem cell populations (Table 1). As shown in Table 1, HCC1395 and MDA-MB-231 cell lines, which express similarly high levels of LBH, both had a large proportion (greater than 90%) of CD44^{high}/CD24^{low} breast cancer stem cells. While HCC1187 did not harbor this high proportion of CD44^{high}/CD24^{low} cells, it had a high proportion of ALDH+ cells relative to the other cell lines that were screened (Table 1). Interestingly, none of the ER positive cell lines harbored high proportions of either type of stem cell, and intermediate expression of LBH correlated with an intermediate proportion of breast cancer stem cells in ER negative cell lines such as BT-549, specifically of the CD44^{high}/CD24^{low} type. Another interesting point is that CD44 itself is actually a target of the Wnt signaling pathway, thus again linking Wnt signaling, LBH, and breast cancer stem cells.

TABLE 1
Summary of LBH Expression and Percentage of CD44+/CD24−
and ALDH+ Breast Cancer Stem Cells in Human Breast
Cancer Cell Lines

| | LBH mRNA | CD44⁺/ | | Cell |
|---|---|---|---|---|
| Cell line | expression | CD24^{-low} | ALDH+ | type |
| ER-Positive | | | | |
| MCF7 | − | 0.70 +/− 0.1 | 2.37 +/− 0.02 | Luminal |
| T47D | − | 0 | 0.52 | Luminal |
| ZR-75-1 | − | 0 | 0.2 | Luminal |
| MDA-MB-361 | − | 0 | 6 | Luminal |
| ER-Negative | | | | |
| HMEC | − | 3.8 +/− 0.29 | 11.9 +/− 0.84 | Basal |
| MCF10A | ++ | 73 +/− 3.24 | 1.24 +/− 0.6 | Basal B |
| MDA-MB-231 | +++ | 93.8 +/− 3.35 | 0.5 +/− 0.1 | Basal B |
| HCC-1395 | +++ | 90.5 +/− 2.31 | 2.02 +/− 0.5 | Basal |
| HCC-1187 | +++++ | 0.54 +/− 0.18 | 8.5 +/− 3.34 | Basal A |
| BT549 | + | 21.04 | 0.7 | Basal B |
| BT20 | + | 6.2 +/− 3.4 | 3.3 | Basal A |

In order to study the function of LBH in breast cancer stem cells, studies that modulate the levels of LBH expression in basal-subtype breast cancer cell lines were conducted by both reduction of expression by RNAi high LBH-positive cell lines and overexpression in LBH-low basal-type cell lines. First, LBH expression in HCC1395 and HCC1187 cells was reduced by RNAi. Using commercially available LBH-specific siRNA (Dharmacon) and protocols recommended by the manufacturer, successful reduction of LBH levels to 10% of mock and control transfected levels was achieved in both HCC1395 and HCC1187 cells. Expression levels on both the RNA and protein levels were monitored at different timepoints post-transfection, and the knockdown remained stable for up to nine days in HCC1395 cells. By maintaining a reduction in LBH levels for a longer period of time, such as nine days, more drastic phenotypic changes should be observed. Following efficient modulation of LBH expression, functional assays were performed to decipher the role of LBH in breast cancer stem cells.

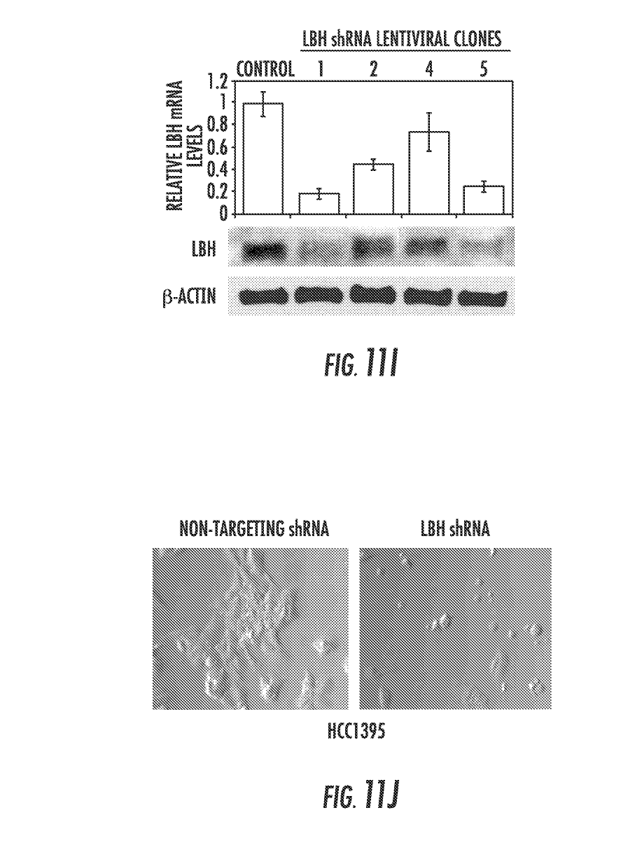

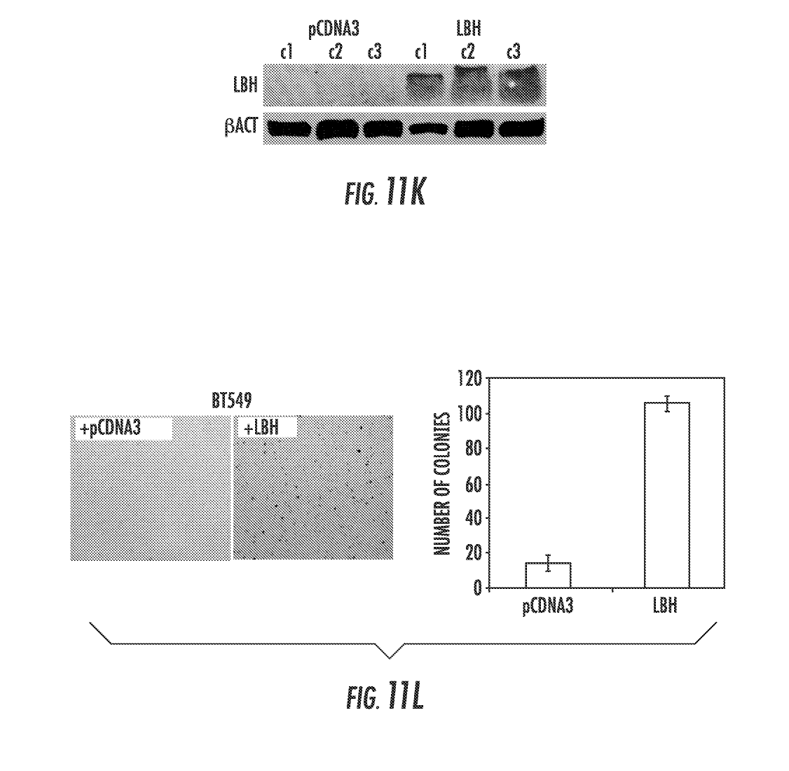

First, the effect of LBH knockdown on the proportion of CD44^{high}/CD24^{low} breast cancer stem cells was examined. For these experiments, 1×10⁶ HCC1395 cells were seeded on 6 cm dishes one day prior to siRNA transfection. Approximately 72 hours post-transfection (approximate time for complete knockdown to occur), the cells were split onto 10 cm dishes to allow for growth and collected 3 or 6 days later for a total knockdown period of 6 or 9 days. Upon harvesting the cells, fractions were taken for protein and RNA, while at least 2×10⁵ cells were used for FACS analysis. For the FACS analysis, the cells were resuspended in 100 μl FACS Buffer (PBS, 2% FBS, 0.1% Sodium Azide) and incubated in the dark with 20 μl CD44-APC and CD24-PE antibodies (BD Bioscience) in the dark for 20 minutes. Cells were washed, resuspended in 500 μl FACS buffer, and analyzed by FACS analysis. Upon knockdown of LBH via siRNA, the proportion of CD44^{high}/CD24^{low} breast cancer stem cells decreased drastically by nearly 25% (red box) after a knockdown period of 9 days (FIG. 11). Conversely, the proportion of CD44^{high}/CD24^{high} cells (yellow box) increased after a period of 9 days of LBH knockdown, indicating that a transition to a more differentiated tumor cell type has occurred (Nieoullon et al., Cell Tissue Res vol. 329:457-467, 2007). CD24 is a differentiation marker for luminal mammary epithelial cells (Sleeman et al., Breast Cancer Res vol. 8:R7, 2006). These experiments were repeated multiple times for both 6 and 9 day knockdowns. Next, stem cell populations will be monitored upon knockdown of LBH in MDA-MB-231 cells to verify these findings in a second cell line. Like HCC1395, this cell line has similar expression of LBH and contains over 90% CD44^{high}/CD24^{low} breast cancer stem cells (Table I). The knockdown and splitting conditions may be modified due to the higher growth rate of these cells, but a similar reduction in the breast cancer stem cell content upon reduction of LBH levels is expected. As an alternative, HCC1187 cells can be used, but would instead require monitoring of ALDH levels for changes in stem cell content. To reciprocate findings, LBH will be overexpressed in several cell lines that normally express low levels of the protein. Two such cell lines are BT-20 and BT-549, which as mentioned above harbor a low to intermediate proportion of CD44^{high}/CD24^{low} cells (Table 1). These cells will be nucleofected with 2 μg linearized pCDNA3 or pCDNA3+N-Lbh vectors according to the manufacturer's protocol (Lonza) and as previously reported with these cells (Wang et al., Mol Cell Biol vol. 25:7953-7965, 2005; Yi et al., Am J Pathol vol. 170:1535-1545, 2007). The proportion of CD44^{high}/CD24^{low} cells is expected to increase in these cells which overexpress LBH.

### Example 2

### Ectopic Expression of Lbh in Normal Mammary Epithelial Progenitor Cells Promotes Self-Renewal and Blocks Terminal Cell Differentiation

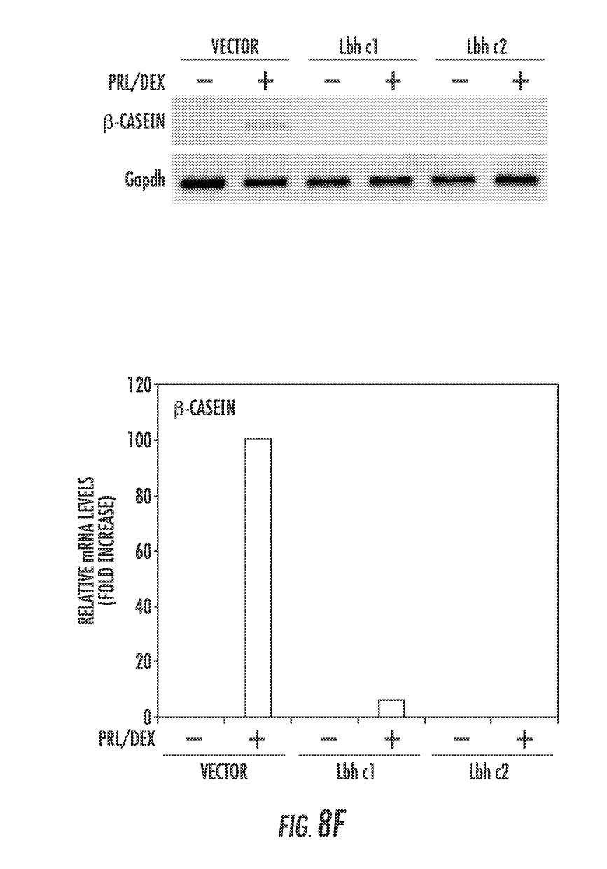

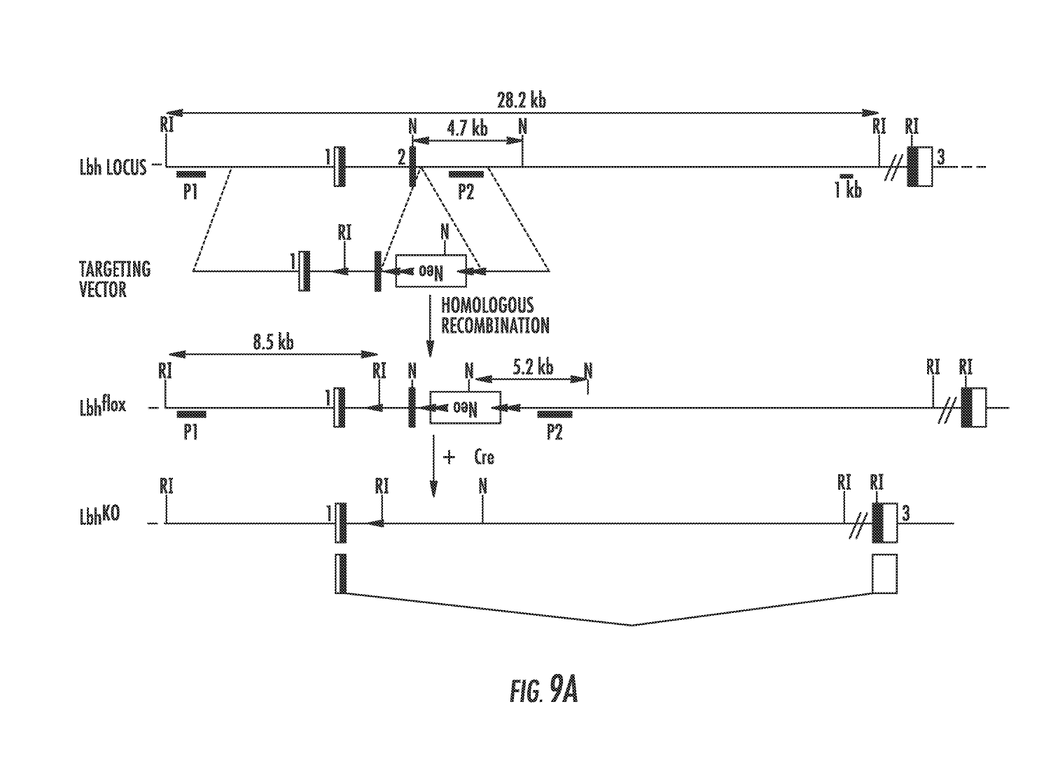

To begin to investigate whether LBH may also affect the differentiation state of normal mammary epithelial cells, LBH was ectopically expressed in the normal immortalized mouse mammary epithelial cell line HC11 (FIG. 8). HC11 cells possess characteristics of mammary epithelial progenitor cells because they have the capability to develop into normal mammary glands when transplanted into cleared fat pads of syngenic host female mice. Moreover, HC11 cells are easy to transfect and represent one of few known non-transformed mammary epithelial cell lines that can be induced to terminally differentiate in 2D in vitro cultures. Apart from observed morphological changes in HC11 cells stably expressing a Flag-tagged LBH, an increase in cell proliferation and a delay in terminal differentiation was observed in different polyclonal LBH-expressing HC11 cell lines as compared to vector-transfected control cell lines (FIGS. 8B, C). Thus, in analogy to fetal cardiomyocytes, LBH overexpression promotes the self-renewal and attenuates the differentiation of mammary epithelial progenitor cells, suggesting that LBH may have a more general role in progenitor cell self-renewal and maintenance.

Based on the above experimental results, a strong case is made that LBH is involved in breast tumorigenesis. There is a clear indication that LBH can promote self-renewal and suppress differentiation of normal and neoplastic mammary epithelial stem cells, a result that is consistent with the activity of this transcriptional regulator in heart development (Briegel et al., Development 132, 3305-3316, 2005). Deregulation of LBH in human breast cancer may be the result of aberrant Wnt signaling activity, as the micrarray data would suggest, but could also occur through genetic instabiliy that is inherent to cancer cells.

### Example 3

### Preparation of Reagents and Development of Assays

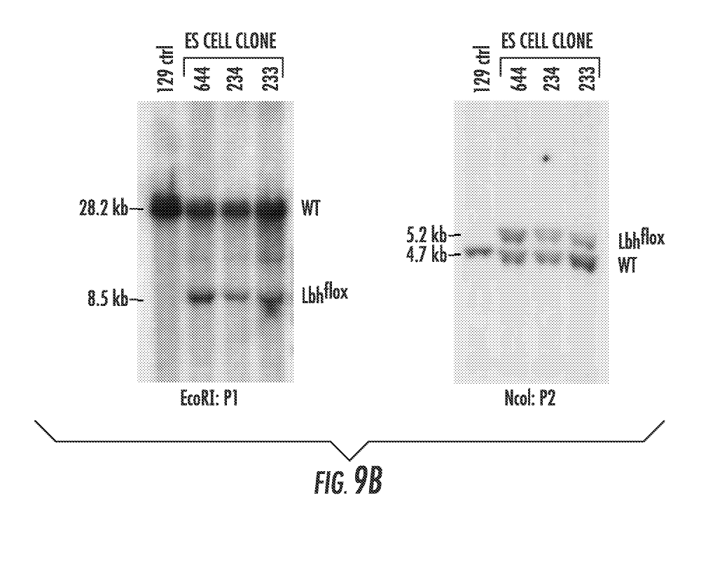

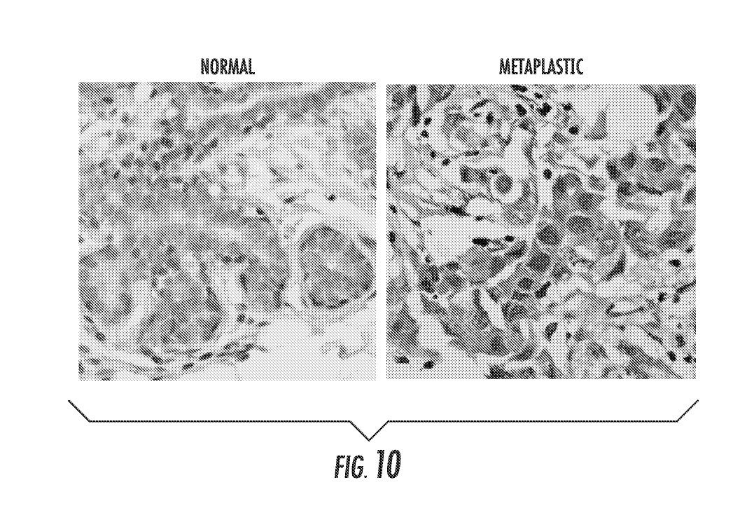

To study the role of Lbh in normal progenitor cell development, a conditional Lbh mouse model was generated (Lbh^{flox} mice). Lbh genomic sequences were isolated from a lgt11-129EV mouse genomic DNA library (Stratagene) using two different Lbh cDNA probes (Briegel and Joyner, Dev Biol 233, 291-304, 2001), and the Lbh gene locus was mapped by restriction enzyme analysis. It was found that the Lbh gene has an unusual genomic structure that is conserved among all vertebrate species. The 105 amino acid (AA) residues of the LBH protein are encoded by three different exons (FIG. 9). These coding exons are separated by intervening sequences of 2.8 kb and 23 kb respectively, suggesting that splicing of LBH must be tightly regulated for functional protein to be produced. The initial attempts to generate a constitutive Lbh gene deletion were not successful. Therefore, a conditional gene targeting strategy was devised based on the Cre-loxP system. To conditionally inactivate the Lbh gene, gene targeting in mouse ES cells was performed to flank exon 2 with two loxP sites (Lbh^{flox} allele). Cre-recombinase-mediated recombination between the two loxP sites will lead to deletion of exon 2 (amino acids 10-43), as well as introduce a frame shift in the coding sequences of the downstream 3^{rd} exon.

This targeting strategy will result in a truncated protein containing only 8 Lbh amino acids. A targeting vector was constructed using BAC recombineering in E. coli, in which a loxP site was inserted into the first intron between exons 1 and 2, and a FRT/loxP site flanked neo cassette was inserted downstream of exon 2 (FIG. 9). This targeting vector was electroporated into 129SV ES cells and ES cell clones with a homologous recombination event were selected in media containing G418. PCR analysis verifying integration of the 3′ homologous arm of the targeting vector identified 4 clones among a total of 288 ES cell clones screened. Southern Blot analysis with external and internal probes confirmed that 3 out of these ES cell clones contained the correct targeting event (FIG. 9). Chimeras were produced from blastocyst injection of two ES cell clones and germline transmission of the targeting event is currently being examined by backcrossing these chimeras into the host strain.

Because LBH is a novel protein, no commercially available antibodies currently exist. Therefore, to generate new tools for Lbh protein detection, a rabbit polyclonal anti-Lbh antibody raised against His-tagged mouse LBH protein purified from E. coli was produced. Antiserum from one of two rabbits tested positive for Lbh-specific antibodies in an ELISA. This antiserum is highly specific for detection of both mouse and human Lbh protein by Western Blot, immunoprecipitation and immunofluorescence analysis on fixed cells and tissue sections.

In preparation for protein-protein interaction studies, several reagents were generated. Bacterial expression vectors expressing Histidine (His) and GST-tagged fusions of the full-length mouse LBH protein (pET28-LBH and pGEX2T-LBH) for in vitro pulldown studies. Recombinant His-tagged LBH protein was produced by expression of pET28-LBH in E. coli and affinity-purified using Ni-agarose. Recombinant GST-tagged LBH protein was produced by expression of pGEX2T-Lbh in E. coli and affinity-purified using Glutathione-coupled agarose beads. Both His-LBH and GST-LBH were contained in the soluble fraction of E. coli cell lysates. To be able to perform reciprocal co-immunoprecipitation studies and for the biochemical identification of LBH protein partners, mammalian expression vectors that express different epitope-tagged versions of the murine LBH protein, including pCDNA3-Flag-LBH (Briegel and Joyner, Dev Biol 233, 291-304, 2001), pCDNA3-HA-LBH and pCDNA-myc-LBH were prepared. These expression vectors were transfected into 293T cells and expression of the different LBH-tagged fusion proteins was assessed by Western Blot analysis using Lbh-specific antibody. Flag and myc-tagged LBH was efficiently expressed, whereas HA-LBH expression vector did not yield a detectable protein. Hence for subsequent biochemical studies Flag-LBH and myc-LBH will be used.

In order to identify LBH interacting proteins in human breast cancer cells, a protocol was devised for co-immunoprecipitation of endogenous LBH protein complexes, which is a modification of published protocols. HCC1187 cells were crosslinked with 1% PFA for 10 min and lysed in RIPA buffer (10 mM Tris pH 7.4, 150 mM NaCl, 1 mM EDTA, 1% TritonX-100, 0.5% Sodium Deoxycholate, 0.1% SDS) containing protease inhibitor cocktail (Sigma) and 5 mM PMSF. 200 μg of total protein extract was pre-cleared with 15 μl Protein A/G Sepharose beads for 2 hours at 4° C. with rotation. Pre-cleared cell lysates were incubated overnight at 4° C. with 1.5 μl anti-Lbh antibody (a-Lbh), or 1.5 μl pre-immune serum (P1). Subsequently, specific protein complexes were isolated by incubation with 15 μl Protein A/G Sepharose for 1.5 hours at 4° C., washing twice for five minutes at 4° C. and elution in SDS-gel loading buffer. Eluted protein complexes were separated by SDS-PAGE. Silver staining of the gel showed faint but enriched protein bands in IP reactions with a-Lbh. Direct coupling of the α-Lbh antibody to Sepharose A beads (Hoijer et al., 1996) is attempted to eliminate contaminating IgG bands. This method is used to isolate protein partners of endogenous LBH in human breast tumor cells.

### Example 4

### Expression, Purification and Structural Characterization of the Novel Transcriptional Regulator LBH

To facilitate research into the mechanistic function of LBH in normal development and disease, protocols for efficient recombinant LBH expression and purification were developed. It was discovered that purified LBH is largely unstructured in solution, as evident by aberrant mobility in both SDS-PAGE and size-exclusion chromatography, low chemical shift dispersion in ¹H-¹⁵N HSQC NMR spectra, and a largely negative band centered around 200 nm in CD spectra. A structurally ‘disordered’ LBH suggests that conformational plasticity may play a crucial role in modulating LBH dependent cotranscriptional processes. Purified recombinant LBH represents an essential reagent for future screening studies aimed at identifying the currently elusive biochemical interactions of LBH that mediate its gene regulator function.

### Materials

The thrombin, AKTAbasic 100 (FPLC), Prepacked GSTrap HP columns (5 mL), the HiPrep 26/10 desalting column, and the Superdex™ 75 16/60 size-exclusion column were from GE Healthcare Biosciences (Piscataway, N.J.). The BL21 Star™ (DE3) protein expression strain of E. coli was from Invitrogen. ¹⁵NH₄Cl was from Cambridge Isotope Laboratories, Inc. (Andover, Mass.). All other chemicals, salts, and buffers were from Sigma, Inc. (St. Louis, Mo.). For preparation of ¹⁵N-isotopic labeled LBH, chromatographic and sample buffers were treated with Chelex, which was from Bio-Rad Laboratories (Hercules, Calif.). All analytical SDS-PAGE were performed on 4-12% gradient Bis-Tris polyacrylamide gels (NuPage), which were developed at 150 V (constant) for ˜1 h, and prior to SDS-PAGE, protein samples in SDS sample buffer were heated at 95° C. for 5 min and cooled on ice.

A full-length LBH cDNA clone from a murine C57BL/6 cDNA library (Image Clone 6813866; BC052470) was obtained from the American Tissue Culture Collection (ATCC). To generate pGEX2T-LBH expression vector, this cDNA clone was used as a template to PCR amplify a 300 bp cDNA fragment (nucleotides 229-546; BC052470), containing the entire LBH protein coding region (amino acids 1-105; Briegel, K. J., and Joyner, A. L. Dev Biol 233, 291-304, 2001) using high fidelity Tgo DNA polymerase (Roche Biochemicals). To incorporate restriction sites at both ends of the LBH coding region a forward primer that contained a BamHI restriction site and a reverse primer that contained an EcoRV restriction site were used. The resultant PCR product was purified and subcloned into a pJET1 vector using the GeneJET PCR cloning kit (Fermentas). Positive subclones were identified by restriction enzyme analysis. Subsequently, a BamHI-EcoRV fragment containing the LBH coding region was doubly digested from the pJET1 plasmid vector and directionally ligated into the BamHI and SmaI restriction sites of pGEX-2T protein expression vector (Promega), which provided an N-terminal GST affinity tag with a thrombin protease cleavage site. The sequence and frame of the insert were confirmed by DNA sequencing.

The pGEX2T-LBH protein expression vector was transformed into the BL21 Star™ (DE3) protein expression strain of E. coli. For routine expression, cells were grown at 37° C. in LB medium containing 100 μg/ml ampicilin and subcultures with an OD600 of 0.5 were induced with 1 mM IPTG for 3 hours. For large scale expression, cells were grown in PG minimal medium (Studier, F. W. Protein Expr Purif 41, 207-234, 2005), which was prepared with the following modifications. First, a desired total volume of 50 mM Na₂HPO₄, 50 mM KH₂PO₄, and 5 mM Na₂SO₄ was mixed and 500-mL aliquots were placed into 2-L baffled-bottom flasks, which were subjected to autoclave sterilization. Immediately before inoculation with starter culture, final concentrations of 2 mM MgSO₄, 56 mM NH₄Cl (1.5 g), 0.6% glucose (3 g), 100 μg/mL carbenicillin antibiotic, and 0.2× of a trace metal mixture were added to each 500 mL containing growth flask. For ¹⁵N-isotopic labeling of LBH, NH₄Cl was replaced with an identical amount of ¹⁵NH₄Cl. A 1000× stock trace metal mixture in 60 mM HCl was prepared as described in Studier (supra) and contained 50 mM FeCl₃, 20 mM CaCl₂, 10 mM MnCl₂-4H₂O, 10 mM ZnSO₄-7H₂O, and 2 mM each of CoCl₂-6H₂O, CuCl₂-2H₂O, NiCl₂-6H₂O, Na₂MoO₄-2H₂O, Na₂SeO₃, and H₃BO₃. A single colony raised from an LB/carbenicillin agar plate was used to inoculate a starter culture of 50 mL PG medium. After growing overnight at 37° C., 15 mL of starter culture was used to inoculate each 500 mL growth flask, which was then allowed to grow at 37° C. to an optical cell density of 0.8 OD₆₀₀.

Protein expression was induced by addition of 0.5 mM isopropyl-β-D-thiogalactopyranoside (IPTG) to cell cultures and allowed to proceed for 4 h at 37° C. In addition, an identical amount of carbenicillin was added to the cultures. The cells (˜3 grams per 500 mL of culture) were harvested by centrifugation for 15 min at 5000 g in an SLA-3000 rotor (Sorvall); and 4 mL/(g cells) of cell wash buffer (50 mM phosphate buffer, pH 7.3, and 500 mM NaCl) was used to re-suspend, wash, and collect by centrifugation a single cell pellet, which was stored at −80° C. The frozen cell pellet was thawed and re-suspended in 10 mL of GST-binding buffer (per gram of cell pellet) containing 50 mM phosphate buffer, pH 7.3, 500 mM NaCl, and 1 mM dithiothreitol. The cells were lysed using the EmulsiFlex-C3 high pressure homogenizer (Avestin, Inc.), and particulates were removed from the lysate by centrifugation for 30 min at 35,000 g in an SS-34 rotor (Sorvall).

The soluble lysate, containing the GST-LBH fusion construct was directly loaded by FPLC (1 mL/min) onto a pair of tandem connected 5 mL GSTrap FF affinity columns equilibrated at 4° C. in GST-binding buffer (see above). The column was first washed with 50 mL of GST-binding buffer containing 0.01% Triton X-100 (1 mL/min). Then detergent was removed by subsequent washing with detergent free GST-binding buffer until the absorbance at 280 nm returned to baseline. GST-LBH was eluted with 50 mM Tris-HCl, pH 8, containing 500 mM NaCl, 2 mM dithiothreitol, and 10 mM glutathione. Fractions containing the GST-LBH construct were combined and exchanged back into GST-binding buffer using a HiPrep 26/10 desalting column. The GST affinity tag was cleaved by direct addition of thrombin protease (10 units of protease per mg of fusion construct) and incubating at 4° C. for 16 h. The cleavage reaction products were directly loaded by FPLC (1 mL/min) onto the tandem GSTrap FF affinity columns, which retained cleaved GST and any uncleaved GST-LBH. The cleaved LBH that was not retained was collected and concentrated to ˜1 mL using Amicon ultrafiltration concentrators (3 kDa molecular weight cutoff) directly loaded by FPLC (1 mL/min) onto a Superdex™ 75 16/60 size-exclusion column equilibrated at 4° C. in 50 mM phosphate buffer, pH 6.5, and 250 mM NaCl. LBH was resolved from all other protein components by isocratic elution with this buffer at 1 mL/min. Fractions containing cleaved recombinant LBH were combined and stored in 10% glycerol at −80° C. LBH protein concentrations were measured by the Bradford Assay.

The amino acid sequence of full length LBH (105 residues) was analyzed for relative amounts of disordered and ordered peptide regions by the PONDR® VL-XT software (Molecular Kinetics, Inc.). PONDR® (predictor of natural disordered regions) is a set of neural network predictors, which utilizes local amino acid composition, flexibility, hydropathy, coordination number, and other factors to score and classify each residue within a sequence as either disordered or ordered. PONDR® VL-XT integrates three feed forward neural networks: the variously characterized long, version 1 (VL1) predictor (Romero et al., Proteins 42, 38-48, 2001), which predicts non-terminal residues, and the X-ray characterized N- and C-terminal predictors (XT) (Li et al., Genome Inform Ser Workshop Genome Inform 10, 30-40, 1999), which predicts terminal residues. Output for the VL1 predictor starts and ends 11 amino acids from the termini. The XT predictors output provides predictions up to 14 amino acids from their respective ends. A simple average is taken for the overlapping predictions; and a sliding window of nine amino acids is used to smooth the prediction values along the length of the sequence. Unsmoothed prediction values from the XT predictors are used for the first and last four sequence positions.

All CD and fluorescence spectra were collected at 25° C. using 10 μM LBH prepared in 50 mM phosphate, pH 6.5, 0.1 mM 2-mercaptoethanol, and either 50 or 250 mM NaCl. CD and fluorescence spectra of the buffer solution were recorded and subtracted from the protein spectra. Equilibrium circular dichroism (CD) measurements were made using the spectropolarimeter on the Bio-Logic Mos450 Stopped-Flow Instrument. The protein far-UV spectra were recorded over a wavelength range of 200-250 nm. For standardization, the baseline spectrum (buffer alone) was subtracted from the spectra, and the results were expressed as mean residue ellipticity [θ]=θ/(c×l×N_{A}); where θ is observed ellipticity (mdeg), c is protein concentration (10⁻⁵ M), l is the optical path length (2 mm), and N_{A} is the number of amino acid residues (108). Fluorescence measurements were made with a Jasco FP-6500 spectrofluorometer using a 5-mm path length cuvette. Emission spectra were acquired from 300 to 500 nm using an excitation wavelength of 295 nm and a 3-nm bandwidth for both excitation and emission.

For NMR analysis, 0.5 mM ¹⁵N-isotopic labeled LBH was prepared in 50 mM phosphate, pH 6.5, 250 mM NaCl, and 5% D₂O. Two-dimensional ¹H-¹⁵N HSQC spectra were collected at 25° C. with a Bruker DMX500 NMR spectrometer (500 MHz for protons) equipped with pulsed-field gradients, four frequency channels, and a triple resonance cryoprobe with an actively shielded z-gradient. For the ¹H-¹⁵N HSQC experiment, the data were recorded by using a pulse sequence in which the HSQC detection scheme was optimized to avoid water saturation (9) and by using the States-TPPI method in the indirect dimension, with a relaxation delay of 1 s. The data were obtained with spectral widths of 1520 and 7000 Hz in f₁ (¹⁵N) and f₂ (¹H), respectively, and with 256 and 1024 complex points, respectively in the t₁ and t₂ dimensions. A total of 8 transients were acquired for each hypercomplex t₁ point with ¹H and ¹⁵N carriers positioned at 4.71 and 120 ppm, respectively. The program NMRPipe was used to process the data. Proton chemical shifts are given with respect to the HDO signal taken to be 4.71 ppm relative to external TSP (0.0 ppm) at 25° C. The ¹⁵N chemical shifts were indirectly referenced.

The coding region of murine LBH (amino acids 1-105) was amplified by PCR from a full-length cDNA clone isolated from a C57/B6 cDNA library and cloned into the expression vector pGEX-2T, which provided an N-terminal GST affinity tag with a thrombin protease cleavage site. After expression at 37° C. in E. coli BL21 Star™ (DE3) cells the majority of the total GST-LBH was found in the soluble fraction. Next, expression of the recombinant GST-LBH fusion construct was optimized in the modified PG minimal medium described above, which enabled large-scale expression and isotopic labeling for NMR spectroscopic characterization. The optimal yield of GST-LBH, expression was achieved by inducing cell cultures (OD of 0.8) with 0.5 mM IPTG for 4 h at 37° C. Cell lysate was collected by homogenization, and GST affinity purification typically yielded 25±3 mg of total soluble protein from 1 L of culture medium (Table 3). A number of protein species co-eluted with the GST-LBH construct, namely endogenous GST (Mᵣ=26 kDa) and one high molecular weight contaminant (Mᵣ=70 kDa; likely Hsp70 chaperone).

Thrombin protease efficiently cleaved the GST-LBH fusion construct, yielding the full length GST affinity tag and full length LBH. In this case, full length LBH includes the additional N-terminal 2 residues as a result of using the pGEX-2T expression vector. This protocol efficiently yielded 5±1 mg of purified full length LBH from 1 L of culture medium (Table 2), which was judged by Coomassie blue staining of 4-12% SDS-PAGE to be of 95% homogeneity.

LBH displayed anomalously faster mobility in Superdex™ 75 size exclusion chromatography (apparent molecular mass of 14 kDa), as well as anomalously slower mobility in SDS-PAGE (apparent molecular mass of 14 kDa), which are ˜1.1-fold higher than the predicted molecular mass of 12.3 kDa calculated from its amino acid sequence (residues 1-105 with N-terminal 2 residues). Aberrant faster mobilities of intrinsically disordered proteins are observed in size-exclusion chromatography, since extended conformations result in larger hydrodynamic dimensions. In addition, it has been pointed out that due to their unique amino acid compositions, intrinsically disordered proteins bind less SDS than globular proteins and therefore show aberrant slower mobilities in SDS-PAGE.

A sequence alignment of LBH proteins from mouse (NP_{—}084275.3), rat (NP_{—}001123352.1), human (NP_{—}112177.2), orangatan (NP_{—}001125165.1), bovine (NP_{—}001092622.1), dog (XP_{—}853968.1), chicken (NP_{—}001026209.1), finch (XP_{—}002198437.1), Xenopus laevis (NP_{—}001081507.1), salmon (ACI34372.1) and zebrafish (NP_{—}956814.1) showed a high degree of conservation (77-90%) of LBH proteins across vertebrate species. The LBH polypeptide (residues 1-105) is highly acidic with a calculated pI of 4.3. Secondary Structure Predictions indicate that only amino acids 95-101 of LBH can fold into an alpha-helix with a probability of 90-100%, whereas the rest of the protein does not have any apparent secondary structure.

Since amino acid sequence analysis of LBH revealed no strong sequence homology with protein sequences of known structure or function, the LBH sequence was subjected to computer analysis using the PONDR® VL-XT software, which utilizes a set of neural network predictors to calculate the probability that amino acid residues exist in either structurally ordered or disordered peptide regions. 70 of 105 LBH residues (66.67%) are predicted to be disordered and are located primarily in two peptide regions spanning residues 15-38 and 60-105. It is interesting to note that the predicted nuclear localization signal (NLS, residues 56-63) overlaps with the most ordered region spanning residues 39-59, whereas the Glutamate-rich putative transcriptional activation domain (residues xx-xx) resides in the longest disordered region. Thus, both secondary and tertirary structure predictions suggest that LBH is largely unfolded. The number of intrinsically disordered proteins (IDPs) fulfilling key biological functions is growing rapidly, and recent studies reveal that they are often involved in regulating molecular recognition and cell signaling. Moreover intrinsic structural disorder was shown to be highly abundant in proteins associated with various human diseases, which is noteworthy given the fact that LBH is implicated in human congenital heart disease as well as in breast cancer.

To evaluate the tertiary structure of LBH, initial biophysical analyses using purified recombinant LBH was performed. The far-UV CD spectra of LBH at 25° C. was determined. The fairly low degree of negative mean residue ellipticity at 222 nm ([θ]₂₂₂=−3272° indicates LBH possesses some fractional amount of secondary structure, albeit far from a completely folded structure. In addition, a fluorescence emission wavelength maximum of 352 nm for the single tryptophan present in LBH (W80) was observed, which is identical to that of free tryptophan in the same buffer (352 nm). Thus, Trp-80 appears to be completely accessible to the aqueous solvent. In order to further assess the relative degree of structural disorder in LBH, it was expressed and purified to contain uniform ¹⁵N-isotopic labeling for two-dimensional ¹H-¹⁵N HSQC NMR analysis, which correlates the ¹H and ¹⁵N chemical shifts (δ) of directly bonded ¹H-¹⁵N pairs (i.e., backbone and side chain amide groups). Multidimensional NMR experiments have the potential to yield residue-specific conformational information of macromolecules in solution, as the backbone amides in folded proteins typically display a broad distribution of nuclear chemical shifts, ranging between ˜7.0-9.5 ppm for protons and between ˜105-135 ppm for nitrogens. For intrinsically disordered proteins, the inherent flexibility of the polypeptide chain and the rapid interconversion between multiple conformations results in poor chemical shift dispersion of most resonances, especially of protons, which narrow to a range between ˜8.0-8.5. The ¹H-¹⁵N HSQC spectrum of uniformly ¹⁵N-labeled LBH was determined. In general, the majority of backbone amide proton resonances exhibited poor chemical shift dispersion (˜7.9-8.7 ppm), indicating substantial regions of structural disorder. In addition, many resonances were broadened, indicating interconversion between multiple conformations occurring within the “intermediate” spectroscopic timescale. Since the amide nitrogen present in the side chains of both glutamine and asparagine contains two bonded protons (—NH₂), this group yields a pair of resonances with the same ¹⁵N chemical shift but different ¹H chemical shifts. Thus, pairs of resonances are observed in the upfield regions (¹⁵Nδ˜113 ppm and ¹Hδ˜6.9 ppm and ˜7.7 ppm), as expected for the −NH₂ group in the side chains of the 3 asparagines and 4 glutamines (N24, 94, 100, and Q43, 88, 92, 105). In addition, a pair of resonances were observed in the downfield regions (¹⁵Nδ=129 ppm and ¹Hδ=10.1 ppm) for the NεH of the aromatic indole side chain of the single tryptophan (W80), indicating slow exchange between two different environments.

TABLE 2
Purification of LBH from E. coli^{a}

| Purification | Volume | Concentration | Yield | Purification |
|---|---|---|---|---|
| (Step) | (mL) | (mg/mL) | (mg) | (fold) |
| Crude lysate | 40 | 11 ± 2^{b} | 440 ± 80 | N/A |
| GST affinity | 11 | 13 ± 2^{b} | 143 ± 22 | 3.1 |
| Superdex ™ 75 | 17 | 1.4 ± 0.2^{c} | 24 ± 4 | 18 |

^{a}All values are reported for purification from expression in 1 L of E. coli.
^{b}Protein concentrations were measured by Bio-Rad protein assay.
^{c}Purified LBH concentrations were measured and converted to mg/mL using the calculated molecular mass.

### Example 5

### LBH, A Novel Wnt Target Gene Deregulated in Breast Cancer

Using a combination of molecular, mammalian tissue culture, mouse genetics and in silico analyses, the molecular pathways operating upstream of Lbh were investigated. In doing so, it was discovered that Lbh expression in epithelial development is tightly controlled by an antagonistic relationship between canonical Wnt/β-catenin and non-canonical Wnt7a signaling. Whereas Lbh transcription is induced by Wnt/β-catenin signaling via four conserved TCF/LEF binding sites in the Lbh gene locus, this induction is efficiently blocked by Wnt7a. It was found that Lbh is aberrantly overexpressed in mammary tumors of MMTV-Wnt1 transgenic mice as well as in highly aggressive basal-subtype human breast cancers. Overexpression of Lbh in HC11 mammary epithelial cells further demonstrates that Lbh suppresses terminal cell differentiation, an effect that could contribute to Wnt-induced tumorigenesis. Collectively, the data described herein suggest that Lbh is a direct Wnt target gene that is reactivated in a particularly lethal form of human breast cancer.

### Materials and Methods

A Lambda gt11-129EV mouse genomic DNA library (Stratagene) was screened with Lbh-specific cDNA probes (Briegel and Joyner, Dev Biol 233, 291-304, 2001). Several overlapping genomic clones comprising approximately 30 kilobases (kb) of the murine Lbh gene locus were isolated and mapped by restriction analysis. A SexAI-NotI genomic fragment containing approximately 1.5 kb of Lbh promoter region and 283 basepairs (bp) downstream of the transcriptional start site including Exon 1 (−1469 to +283) was inserted into the XhoI-HindIII sites of a pGL3-Luciferase vector (Pwt). Lbh enhancer regions 1 and 2 (−6365 to −6445 and +1240 to +2003, respectively) were PCR amplified and cloned individually into the KpnI site of the Pwt plasmid construct upstream of the Lbh promoter to generate constructs E1 wt and E2 wt. In vitro mutagenesis was performed to introduce mismatch mutations into Lbh-specific TCF/Lef binding elements (TBEs) T1-T4 using the QuikChange II XL Site-Directed Mutagenesis Kit (Stratagene).

For quantitative Real-Time PCR (qPCR), cDNA was synthesized from 1 μg DNase-treated total RNA according to the manufacturer's protocol using the Transcriptor First Strand cDNA Synthesis Kit (Roche). qPCR reactions were carried out in 20 μl using SYBR Green Master Mix (NEB) containing 10 nM of 6-carboxyfluorescein (Sigma) as a reference dye along with sample, primers, and water. Primer sequences and thermocycling protocol are available upon request. The reactions were performed in triplicates on a Biorad iCycler and quantified using the iCycler iQ software. The relative quantities of LBH, DKK1 and β-catenin/CTNNB1 mRNA were determined for each sample based on the Ct value and normalized to the corresponding values of the housekeeping gene GAPDH. In the methods described herein, qPCR can be used as a diagnostic tool to detect LBH-expressing tumor cells in blood samples or biopsies or other clinical cell/tissue samples that are limited in size. The primers for LBH qPCR are as follows. Primer 1 (sense strand of human LBH gene): TCACTGCCCCGACTATCTG (SEQ ID NO:10), and Primer 2 (antisense strand of human LBH gene): GGTTCCACCACTATGGAGG (SEQ ID NO:11).

To generate a His-TCF4 expression vector, the TCF4 DBD (DNA-binding domain residues 265-496) was PCR-amplified using pGST-TCF4 (Niida, Oncogene 23, 8520-8526, 2004) as a template and inserted into the BamHI-HindIII restriction sites of pET28A vector (Novagen). Recombinant His-TCF4 was expressed in E. coli and purified with Nickel-beads (Novagen) according to the manufacturer's protocol.

Double-stranded DNA oligonucleotides (30 mers) containing the genomic TBE sites T1-T4 with flanking sequences were 5′ end-labeled with ³²P and 5000 cpm/μl of labeled probe was incubated with 1 μg of recombinant His-TCF4 protein in a total volume of 15 μl binding buffer. For competition and supershift experiments, His-TCF4 was pre-incubated with unlabelled DNA oligonucleotides at 400-fold excess or with 1-5 μg of anti-6× His tag antibody (Abcam) for 10 minutes prior to addition of labeled probe. Samples were separated on 5% non-denaturing polyacrylamide gels for 1 h at 400V. Protein-DNA complexes were detected by phosphoimaging on a Storm 840 Scanner (Molecular Dynamics).

Luciferase reporter assays were performed as described in Briegel et al. (Development 132, 3305-3316, 2005) with the following modifications: one day prior to transfection 2.0×10⁵ cells were plated per well of a 12-well plate. Cells were co-transfected with 100 ng of different luciferase reporter plasmids (Pwt, E1wt, E2wt or TOPFlash, FOPFlash) and 300 ng of pCDNA/β-cateninS37Y expression plasmid using Lipofectamine 2000 reagent (Invitrogen). The fold transactivation of each Lbh-luciferase construct represents the ratio between normalized luciferase values of β-cateninS37Y co transfected cells and of cells transfected with the respective Lbh-luciferase constructs alone. For TOPFlash reporter assays, fold activation represents the ratio between normalized TOPFlash and FOPFlash activities. All transfections were performed in duplicates, and results of at least three independent experiments were statistically analyzed using a paired Student's t-test.

HMEC cells were obtained from Clonetics, all other human breast epithelial tumor cell lines were from the American Type Culture Collection (ATCC) and grown per recommendations of these distributors. 293T and L-Wnt3a cells (ATCC) were cultured in DMEM medium containing 10% FBS and grown under standard conditions at 37° C. in 5% CO₂ atmosphere. Wnt3a conditioned medium was prepared according to the distributor's protocol. HC11 cells were grown in RPMI medium supplemented with 10% FBS, 10 ng/ml Insulin (Sigma) and 5 μg/ml EGF (Invitrogen). Stable polyclonal cell lines were established by Lipofectamine transfection of HC11 cells with linearized pCDNA3 empty vector or pCDNA3-NLbh plasmid followed by selection in 200 μg/ml of G418 (Invitrogen). Wnt Induction and RNAi. For time course experiments, 293T cells were co-cultured with Wnt3a-conditioned medium for 0, 4, 8, 16 and 24 h. Inhibition experiments used 100 ng/ml of recombinant human DKK1, Wnt5a, or Wnt7a (R&D Systems), which were either added alone for the indicated time points, or 8 h prior to an 8 h treatment of cells with Wnt3a-conditioned media. For RNAi studies, 4×10⁵ of 293T cells were transfected with 100 nM of synthetic siRNA specific for CTNNB1/β-catenin or a scrambled control sequence using Dharmafect 1 reagent (Dharmacon). Approximately 65 h after siRNA transfection, 293T cells were trypsinized and transferred to a dish with twice the surface area to allow for growth. 72 h post-transfection, Wnt3a conditioned media was added for an additional 16 h. After harvesting the cells, total RNA was isolated using TRIzol® Reagent (Invitrogen) and treated with Turbo DNase (Ambion). cDNA was synthesized from 1 μg DNase-treated total RNA using the Transcriptor First Strand cDNA Synthesis Kit (Roche). qPCR reactions were carried out in 20 μl using SYBR Green Master Mix (NEB) containing 10 nM of 6-carboxyfluorescein (Sigma) as a reference dye, 50-100 ng of cDNA and 2 μM of primers. The reactions were performed in triplicates on a Biorad iCycler and quantified using the iCycleriQ software. The relative quantities of LBH, DKK1 and β-catenin mRNA were determined for each sample based on the Ct value normalized to the corresponding values for GAPDH. Mice, Histology, In Situ Hybridization (ISH) and X-gal Staining. MMTV-Wnt1 (B6SJL-Tg(Wnt1)1Hev/J) and TopGal (Tg(Fos-lacZ)34Efu/J) transgenic mice were purchased from Jackson Laboratories (Bar Harbor, Me.).

Whole mount in situ RNA hybridization and X-gal staining of embryos and sections were performed as previously described (Briegel and Joyner, Dev Biol. 233:291-304, 2001). Moreover, 14 μM cryosections of snap-frozen mouse mammary glands or 5 Mm paraffin sections of MMTV-Wnt1 mammary tumors were hybridized with a mouse Lbh-specific anti-sense probe (Briegel and Joyner, Dev Biol. 233:291-304, 2001).

Mammary epithelial cells (MEC) from mammary glands of wild type mice were isolated via proteolytic digestion with 100 units/ml hyluronidase (Sigma) and 2 mg/ml collagenase A (Roche) in 15 ml DMEM for 3 h at 37° C. with gentle agitation followed by washing in DMEM plus 5% FBS. Tumors from MMTV-Wnt 1 transgenic mice were snap frozen in liquid nitrogen and mechanically pulverized. Isolated MEC and ground tumors were lysed in RIPA lysis buffer (20 mM Tris pH7.5, 150 mM NaCl, 1% NP-40, 0.5% Sodium Deoxycholate, 1 mM EDTA, 0.1% SDS) containing protease inhibitors (Amresco). For Western blot analysis a total of 25 μg protein extract per sample was separated by SDS-PAGE, blotted on nitrocellulose membrane and incubated with the following antibodies in TBST (20 mM Tris HCl pH 7.5, 140 mM NaCl, 0.1% Tween 20) plus 5% non-fat dry milk: a rabbit polyclonal Lbh antibody raised against murine Lbh and purified by Melon Gel IgG purification (Pierce) (1:1,000), polyclonal Keratin 5 (Covance; 1:10,000), polyclonal Keratin 8/18 (Progen; 1:2,000), monoclonal β-actin (AC-15, Sigma A5441; 1:50,000), and anti-rabbit, anti-mouse, or anti-guinea pig HRP-coupled secondary antibodies (Amersham, Sigma; 1:10,000). Immunofluorescence. Cells were grown overnight on BD Bioscience Culture Slides at a density of 2×10⁵ cells per well and induced with Wnt3a conditioned medium for 6 h. Cells were fixed with 2% paraformaldehyde in PBS for 15 min at room temperature, followed by cell permeabilization in 0.3% Triton X-100 in PBS. Cells were blocked for 1.5 h in PBS plus 10% Normal goat serum (NGS) and incubated with β-catenin antibody (BD Biosciences; 1:200) followed by subsequent incubation with anti-mouse Cy3 (Jackson ImmunoResearch; 1:400). Cells were mounted in Slowfade plus DAPI (Molecular Probes) according to the manufacturer's protocol. Images were taken on a DMRI Leica Inverted Microscope.

Chromatin Immunoprecipitation (ChIP) was performed as follows. HC11 cells were grown to 70% confluence prior to addition of Wnt3a conditioned medium for 3 h. Cells were fixed in a final concentration of 1% formaldehyde for 10 min at room temperature followed by a quenching of fixative with 125 mM Glycine. Cells were incubated for 10 min on ice in swelling buffer (5 mM PIPES pH 8.0, 85 mM KCl, 1% NP-40) at a concentration of 5×10⁷ cells/ml followed by dounce homogenization 15 times. Nuclei were pelleted at 2,500 rpm for 5 min and resuspended in sonication buffer (50 mM Tris-HCl pH 8.0, 10 mM EDTA, 1% SDS) at a concentration of 1×10⁸ cells/ml. Sonication of cells for 6 pulses of 15 sec each on ice/water at 50% power on a Misonix sonicator resulted in chromatin fragments of an average length of 1 kb. Lysates were cleared for 10 minutes at top speed. For each IP reaction, 1×10⁷ cell equivalents were diluted to 1 ml total volume in dilution buffer (0.01% SDS, 1.1% TritonX-100, 1.1 mM EDTA, 16.7 mM Tris-HCl pH 8, 167 mM NaCl) and pre-cleared for 2 h with 40 μl Protein A/G sepharose beads (GE Healthcare). Cleared lysates were incubated overnight with 5 μg of normal rabbit IgG, antiacetyl Histone 3 or anti-β-catenin antibodies (Upstate). Thereafter, precipitation of immunocomplexes was performed according to the Upstate EZ ChIP protocol. PCR reactions for 35 cycles were carried out using Phusion polymerase (Finnzymes).

For proliferation assays, 2,000 cells were seeded in triplicates on 96 well plates. Two hours post-plating, 20 μl of CellTiter 96 AQueous One Cell Proliferation Assay reagent (Promega) was added to wells containing cells and blank media controls. Reagent was applied at the same time daily and absorbance at 492 nm was measured 2 h later on a microplate reader for 7 days. Background was eliminated by subtracting values of media controls. Differentiation of HC11 cells was carried out for 3 days according to Ball et al. (Embo J., 7:2089-2095, 1988).

Affymetrix gene expression data representing a total of 1,107 primary breast tumors from six previously published microarray studies (Chin et al., Cancer Cell 10:529-541, 2006; Desmedt et al., Clin Cancer Res 13:3207-3214, 2007; Ivshina et al., Cancer Res 66:10292-10301, 2006; Pawitan et al., Breast Cancer Res 7:R953-964, 2005; Sotiriou et al., J Natl Cancer Insti 98:262-272, 2006; Wang et al., Lancet 365:671-679, 2005) were integrated as described previously using a mean-batch centering method (Sims et al., BMC Medical Genomics 1:1-14, 2008; Ben-Porath et al., Nat Genet 40:499-507, 2008). Centroid prediction (Calza et al., Breast Cancer Res 8:R34, 2006) was used to assign the tumors from each dataset to the five Norway/Stanford subtypes (Basal, Luminal A, Luminal B, ERBB2 and Normal-like (Perou et al., Nature 406:747-752, 2000; Sorlie et al., Proc Natl Acad Sci USA 98:10869-10874, 2001; Sorlie et al., Proc Natl Acad Sci USA 100:8418-8423, 2003). Centered average linkage clustering of the integrated tumor datasets was performed using the Cluster (Eisen et al., Proc Natl Acad Sci USA 95:14863-14868, 1998) and TreeView programs as described previously (Sorlie et al., Proc Natl Acad Sci USA 98:10869-10874, 2001).

### RESULTS

To elucidate the molecular pathways acting upstream of Lbh, murine Lbh genomic sequences were screened for potential transcription factor binding sites. This in silico search identified four conserved putative TCF/LEF-binding elements (TBEs) in the Lbh gene locus. Two TBEs with the consensus motif 5′-CTTTG(A/T)(A/T)-3′ were located within an enhancer region (E1)-6245 (T1) and −6195 (T2) base pairs (bp) upstream of the Lbh transcriptional start site (FIG. 1A). In addition, two consensus TBEs were found in an enhancer (E2) contained within the first intron of the Lbh gene at positions +1558 (T3) and +2145 (T4) by (FIG. 1A). To directly assay for TCF binding to these sites, electrophoretic mobility shift analysis (EMSA) was performed. Recombinant TCF4 protein bound with high affinity to all Lbh-specific TBEs (T1-T4), but not to an unspecific oligonucleotide (FIG. 1B). TCF4 binding to these sites was efficiently competed by addition of 400-fold excess of unlabeled wild-type (+) oligonucleotide as well as increasing amounts of an antibody against recombinant TCF4 protein, but not by addition of 400-fold excess of mutant (m) oligonucleotide (FIGS. 1B, C). Subsequently, cell-based reporter assays were performed to test whether the Lbh genespecific TBE sites (T1-T4) were functionally responsive to overexpression of β-catenin, which is the Wnt-inducible component of the TCF/β-catenin transcriptional complex. HC11 mouse mammary epithelial cells were used because this cell line abundantly expresses TCF4, but has low endogenous Wnt/β-catenin signaling activity. The Lbh enhancer regions E1 and E2 were cloned individually into a promoter-luciferase construct (Pwt) upstream of approximately 1.5 kb of murine Lbh gene promoter sequences that do not contain any apparent consensus binding sites for TCF/β-catenin. The three Lbh-Luciferase reporter constructs (Pwt, E1 wt and E2 wt) were co-transfected with a pCDNA plasmid vector expressing constitutively active β-catenin (β-cateninS37Y). As shown in FIG. 1D, Lbh-luciferase constructs containing wild-type TBE sites (E1wt, E2wt) were induced by β-cateninS37Y approximately 14-18 fold. The basal Lbh promoter-Luciferase construct (Pwt) also showed transcriptional activation despite the lack of TBEs, indicating that β-catenin may also have indirect effects on Lbh promoter activity. Most importantly, however, mutations of both T1 and T2 together (E1t1-2), or of T3 and T4 either individually or in combination (E2t3, E2t4 and E2t3-4) significantly reduced (2.5-3 fold; p<0.02) transcriptional activation of Lbh reporters by β-cateninS37Y (FIG. 1D). Mutation of either T1 or T2 alone had little effect, suggesting that binding of a β-catenin/TCF4 transcriptional complex to only one of these sites is sufficient for activity of this enhancer (E1). These data suggest that Lbh is activated by the canonical Wnt pathway at the transcriptional level via high affinity TCF-binding elements located within upstream and intronic enhancer regions of the Lbh gene.

To further test whether LBH is a bona fide Wnt/β-catenin target gene, whether or not endogenous LBH mRNA expression was responsive to Wnt was examined. Human 293T embryonic kidney epithelial cells were co-cultured with Wnt3a conditioned medium (hereafter referred to as Wnt3a), and mRNA levels of LBH, as well as of a known Wnt target gene, DKK1 (Chamorro et al., EMBO J 24:73-84, 2005), were assayed over a 24 hour time course using qPCR analysis. Induction of LBH was detectable within 4 h of Wnt3a treatment and reached a maximum at 16 h (>4 fold increase; FIG. 2A). DKK1 was induced to a smaller degree and its induction was delayed as compared to LBH (FIG. 2A). Induction of both LBH and DKK1 mRNA expression by Wnt3a was efficiently blocked by recombinant DKK1 protein (FIGS. 2A, C), a potent inhibitor of canonical Wnt/β-catenin signaling. Moreover, Wnt3a-mediated induction of LBH and DKK1 was abrogated by depletion of β-catenin expression using RNAi, while scrambled control siRNA had no effect (FIG. 2B). These results reinforce the notion that LBH is a direct transcriptional target of the canonical Wnt signaling pathway.

To investigate whether Wnt ligands that signal through non-canonical pathways could also induce LBH gene expression, 293T cells were treated for 16 h with recombinant Wnt5a or Wnt7a (FIG. 2C). In contrast to Wnt3a, both Wnt5a and Wnt7a treatment alone did not induce LBH, but modestly reduced baseline LBH and DKK1 expression (FIG. 2C). Since Wnt5a has previously been shown to inhibit Wnt3a-induced canonical Wnt signaling, LBH gene expression in cells treated with both Wnt3a and the individual noncanonical Wnt ligands was examined. Surprisingly, Wnt7a strongly inhibited LBH and DKK1 induction by Wnt3a, whereas Wnt5a failed to block Wnt3a-mediated induction of these genes (FIG. 2C). Thus, LBH is specifically induced by canonical Wnt signaling, whereas non-canonical Wnt7a signaling has an antagonistic effect on LBH expression and its induction by Wnt3a.

To test the hypothesis that LBH might be implicated in Wnt-induced tumorigenesis, Lbh expression was examined in MMTVWnt1 transgenic mice, a mouse model for Wnt-induced breast cancer (Tsukamoto et al., Cell 55:619-625, 1988). Moreover, since the expression pattern of Lbh in normal adult breast tissue was not known, Lbh expression during postnatal mouse mammary gland development was analyzed using RNA in situ hybridization and Western blot analyses. In post-pubertal (7 weeks) virgin female mammary glands, expression of Lbh was restricted to stromal, basal-myoepithelial, and terminal end bud (TEB) mammary epithelial cells. In contrast, Lbh was absent from ductal luminal mammary epithelial cells at all postnatal development stages analyzed. During pregnancy, Lbh levels drastically increased and Lbh transcripts were primarily detected in the proliferating lobuloalveolar compartment, a pattern that was maintained during early involution. Notably, Lbh expression was virtually absent in lactating mammary glands, suggesting that Lbh is not expressed in terminally differentiated secretory mammary epithelial cells. Most remarkably, Lbh expression levels were significantly elevated (2.8-4.2 fold) in 9 out of 10 mammary tumors from different MMTV-Wnt1 transgenic mice as compared to non-pregnant mammary glands, HC11 cells, and mammary epithelial cells isolated from equiparous wild-type littermates. Moreover, in MMTV-Wnt1 tumors, which phenocopy human basal breast cancer (Herschkowitz et al., Genome Biol 8:R76, 2007), Lbh expression correlated with expression of the basal marker Keratin 5, whereas it inversely correlated with expression of the luminal markers Keratin 8/18. Thus, Lbh is expressed at normal levels in basal and proliferative alveolar mammary epithelial cells during normal mammary gland development, whereas it is overexpressed in Wnt-induced breast epithelial tumors.

Lbh overexpression suppresses the differentiation of HC11 mammary epithelial cells. As Lbh expression was found specifically in cellular targets of canonical Wnt signaling during normal mammary gland tissue homeostasis and that Lbh is upregulated in Wnt-induced mammary tumors, the functional relationship between Wnt/β-catenin signaling and Lbh was further investigated in a cell culture system for mammary epithelial development. HC11 was chosen, because it is one of few existing non-transformed mammary epithelial cell lines that can be induced to differentiate in vitro with lactogenic hormones. Moreover, overexpression of different Wnt ligands has been shown to lead to cellular transformation of these cells. To test whether Lbh could be downstream of canonical Wnt signaling in mammary epithelial cells, HC11 cells, which do not express Lbh, were treated with Wnt3a. As shown in FIG. 9, Wnt3a treatment resulted in nuclear localization of β-catenin as well as a rapid increase in Lbh mRNA levels (FIGS. 8A, B). In addition, ChIP analysis showed that the Lbh gene regulatory sequences T1-T4 (FIG. 1A) were occupied by endogenous β-catenin in Wnt3a-treated cells, but not in untreated control cells (FIG. 8C).

Having demonstrated that Lbh is a direct transcriptional target of Wnt/β-catenin in HC11 cells, whether or not overexpression of Lbh elicits some of the same effects that have been reported for overexpression of Wnt ligands in this cell line was investigated. Several polyclonal HC cell lines stably expressing Lbh (Lbh c1 and c2) were generated by transfection with a pCDNA3-Lbh plasmid, and Lbh overexpression was confirmed by qPCR and Western Blot analyses (FIG. 8D). No Lbh expression was detectable in vector control transfected, or in the parental HC11 cells (FIG. 8D). Although ectopic Lbh expression did not result in cell transformation as determined by soft agar assays, the growth rates of Lbh-expressing HC11 cells were significantly increased as compared to vector control cells (FIG. 8E). Moreover, whereas differentiation induction with prolactin and dexamethasone increased mRNA expression of the milk protein β-casein in parental and vector control cells, induction of β-casein in response to these lactogenic hormones was lost in HC11-Lbh cells (FIG. 8F). Thus, overexpression of Lbh promotes cell proliferation and blocks terminal differentiation of HC11 mammary epithelial cells.

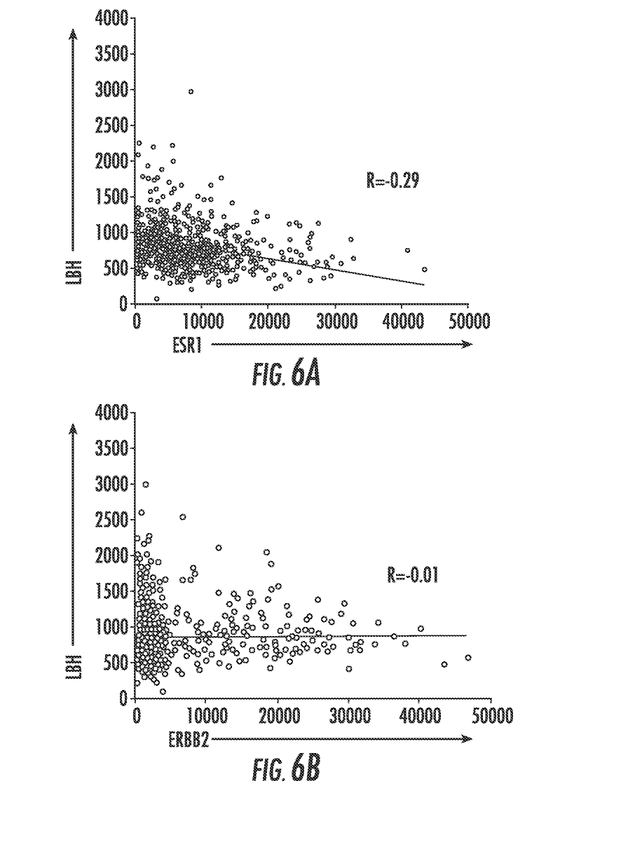

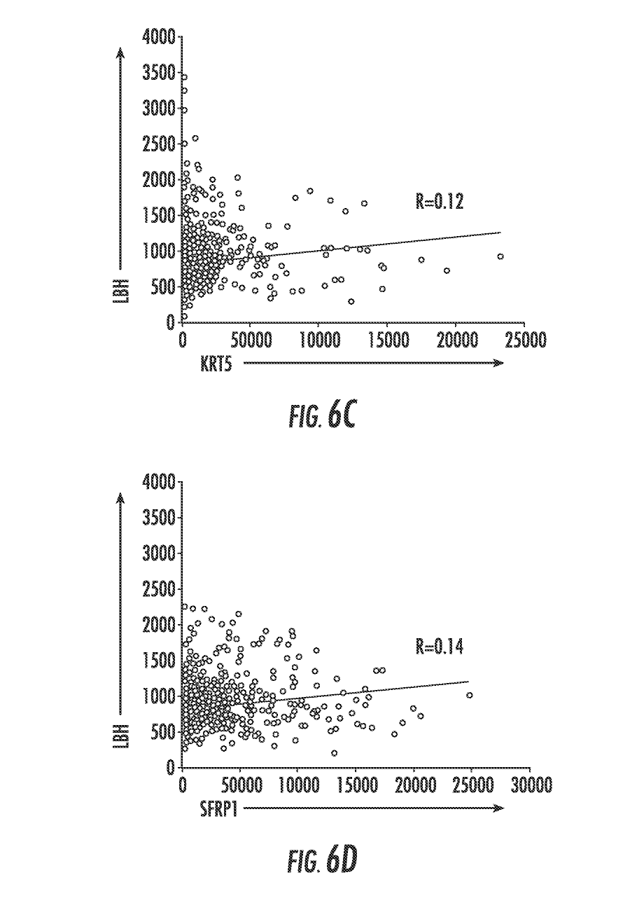

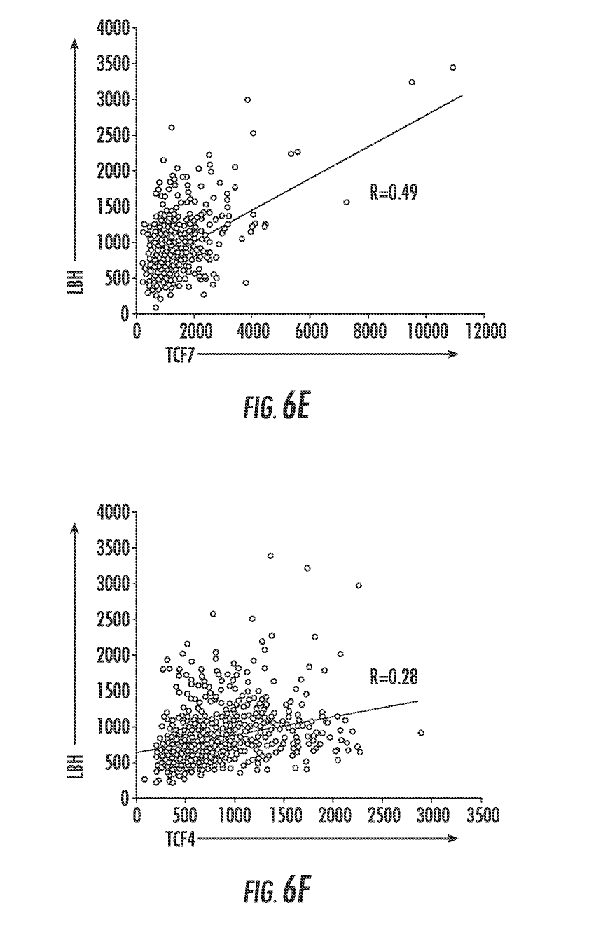

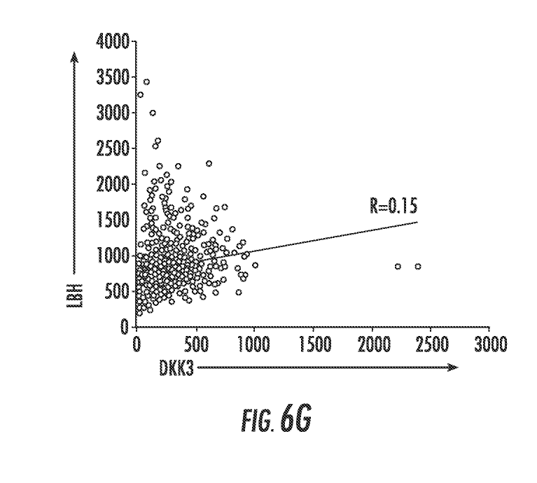

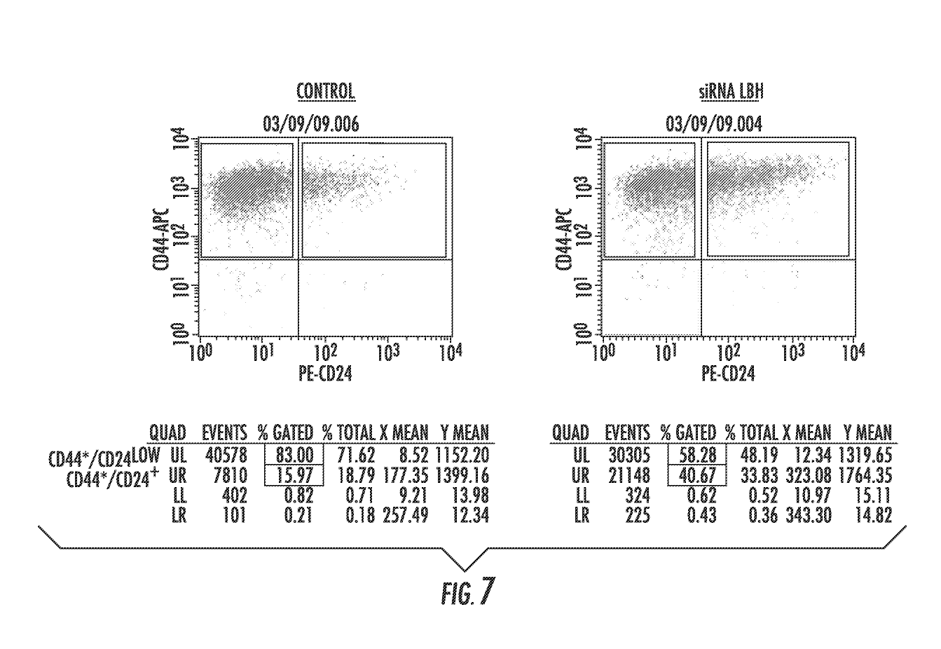

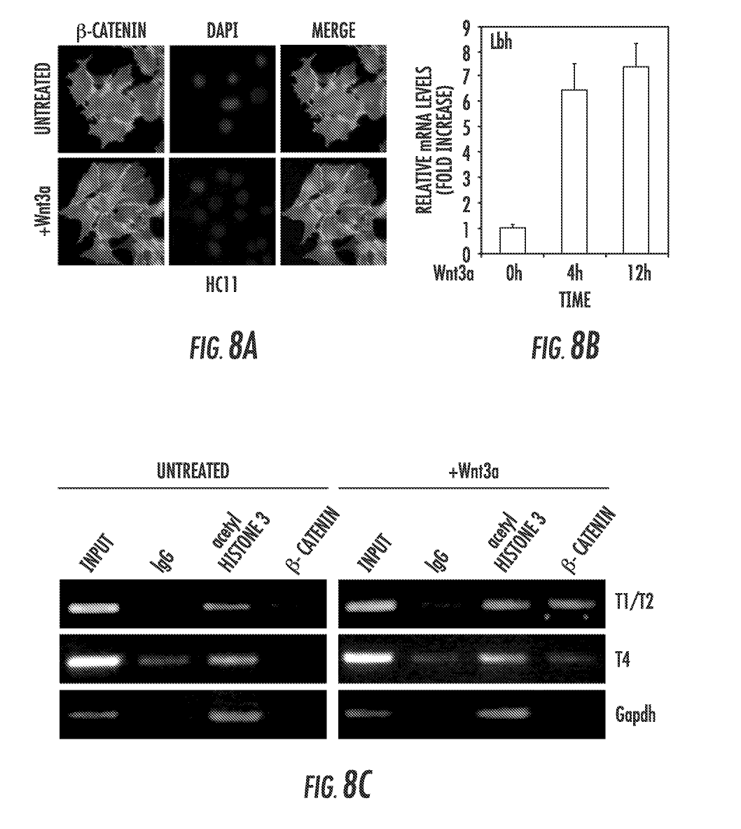

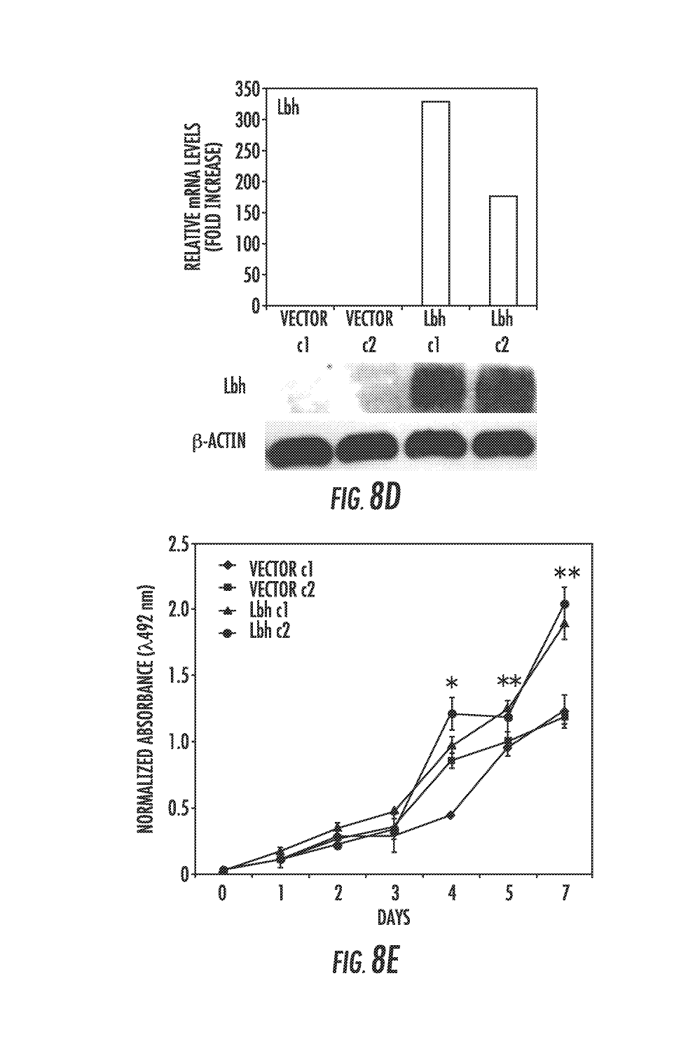

To further examine whether LBH might be deregulated in human breast cancer, metaanalysis of six Affymetrix gene expression datasets comprising 1,107 primary human breast cancers was performed as previously described (Sims et al., BMC Medical Genomics 1:1-14, 2008). These data represent the five ‘intrinsic’ breast tumor subtypes Normal-like, Luminal A, Luminal B, ERBB2-positive and Basal-like (Sortie et al., Proc Natl Acad Sci 98:10869-10874, 2001), which can be distinguished by specific gene signatures and differences in clinical outcome, with Basal-like breast cancers having the worst prognosis (Perou et al., Nature 406:747-752, 2000; Sortie et al., Proc Natl Acad Sci 98:10869-10874, 2001). Strikingly, LBH expression was significantly associated with aggressive, poorly differentiated basal-type carcinomas. Almost half (45%) of the basal breast tumors had high LBH expression levels. In contrast, elevated LBH was observed in far smaller proportions of Normal-like (24%), Luminal A (16%), Luminal B (23%) and ERBB2+ (27%) breast carcinomas. Moreover, a strong inverse correlation was observed between LBH and estrogen receptor alpha (ESR1) expression, FIG. 6; R=−0.29, p<0.0001), whereas no significant correlation existed with ERBB2 status (FIG. 6; R=−0.01). Most remarkably, however, LBH expression in breast tumors strongly correlated with the basal marker Keratin 5 and canonical Wnt pathway genes, such as SFRP1, TCF4, TCF7 and DKK3 (FIG. 6). This figure shows a correlation with clinical breast cancer markers and WNT (p<0.0001). These data highlight LBH as a novel molecular marker for difficult-to-treat ER-negative basal-type breast cancer and suggest that LBH deregulation in breast cancer could be a consequence of oncogenic Wnt signaling.

Human breast carcinoma cell lines were analyzed to validate the findings. Published Affymetrix gene expression data from 51 human breast cancer cell lines was first queried to confirm the existence of a relationship between LBH expression and breast cancer subtype. Expression of LBH was significantly higher in both the ‘Basal A’ and ‘Basal B’ cell line subtypes than those classified as ‘Luminal’ (p<0.007; FIG. 4A). This figure shows that WNT7A inhibits LBH and canonical WNT signaling in triple-negative breast cancer cells. Specifically, 50% of the Basal A (n=12) and 29% of the Basal B (n=14) cell lines had high (upper quartile) expression of LBH, compared to only 12% of Luminal (n=25) cell lines (FIG. 4A). Similar results were observed in a more recent cDNA microarray study of breast cancer cell line gene expression ((Kao et al., PLoS One 4:e6146, 2009); FIG. 4A), which also demonstrated significantly higher expression of LBH in the Basal A and B than the Lumina (cell lines (42%, 40% and 12% with high expression respectively). Next, LBH expression was examined in a panel of 13 established human breast cancer cell lines using qPCR and Western Blot analysis. High levels of LBH expression were only detected in the ER-negative basal subtype breast tumor cell lines HCC1395, MDA-MB-231 and HCC1187 (FIGS. 4B, D). In contrast, none of the ER-positive lines (MCF7, T47D, ZR-75-1, MDA-MB-361) or ER-negative (SK-BR-3) luminal cell lines, expressed LBH at detectable levels (FIGS. 4B, D). Furthermore, LBH protein was not detected in finite lifespan human mammary epithelial cells (HMEC) or in non-malignant MCF10A cells (FIG. 4D). Thus, consistent with the gene expression analysis in primary breast tumors, LBH expression in breast cancer-derived cell lines correlated with an invasive basal carcinoma phenotype and inversely correlated with expression of the good prognostic marker ER. LBH deregulation in breast cancer may be due to aberrant Wnt/β-catenin pathway activation. To begin to investigate the mechanisms underlying LBH deregulation in breast cancer, comparative genomic hybridization array (aCGH) data that were available for these breast tumor cell lines was queried. Only one of three LBH-overexpressing basal tumor cell lines (HCC1395) had a modest increase in LBH copy number (FIG. 4C). Moreover, aCGH analysis of primary breast tumor data sets did not show a significant correlation between increased LBH copy number and LBH overexpression in basal subtype tumors, suggesting that changes in LBH gene dosage play a minor role in LBH dysregulation in basal breast carcinomas.

To further test whether LBH overexpression may be a consequence of aberrant Wnt signaling, endogenous Wnt signaling activity was examined in LBH-positive breast tumor cell lines using TOPFlash reporter assays. Strikingly, 2 out of 3 of these cell lines (HCC1187 and HCC1395) displayed increased Wnt/β-catenin signaling activity similar to HC11 cells transfected with pcDNA/β-cateninS37Y (FIG. 4E). No detectable Wnt activity was measured in MDA-MB-231 cells, nor in HC11 cells, which served as a negative control. Furthermore, treatment of HCC1395 cells with DKK1 inhibitor blocked LBH expression, indicating that expression of LBH in this breast tumor cell line is dependent on Wnt/β-catenin signaling (FIG. 4F). Finally, whether Wnt7a could serve as a means to inhibit LBH expression in basal breast tumor cells was explored. Remarkably, treatment of HCC1395 cells with Wnt7a efficiently suppressed mRNA expression of LBH as well as of DKK1 (FIG. 4F). Thus, aberrant canonical Wnt signaling, at least in part, is responsible for LBH overexpression in basal subtype breast carcinoma cells. Furthermore, WNT7A provides an efficient means to inhibit LBH expression and canonical WNT signaling in basal breast cancer cells.

### Example 6

### LBH Expression in Breast Cancer Cells and Effects of LBH Deregulation On Breast Cancer Stem Cell Development

As described above, LBH is expressed at abnormally high levels in ‘triple’ (ER-estrogen receptor, PR—progesterone receptor and Her2-Heregulin-2)-negative breast cancers as a consequence of WNT pathway hyperactivation. Triple negative breast cancers (TNBC) are characterized by an advanced-grade, poorly differentiated, basal-subtype tumor phenotype with a ‘high’ CSC contribution. Since aberrant activation of the WNT signaling pathway through deregulation of downstream targets is a major transforming event leading to formation of tumor-initiating and treatment resistant CSC with high metastatic potential, the hypothesis that deregulation of LBH in breast tumors enhances the self-renewal and maintenance of breast cancer stem cells was tested.

Towards investigating to what extent LBH is expressed in breast CSC, using FACS analysis, the proportion of CD44^{high}/CD24^{low} or ALDH+ tumor stem cell populations in LBH-expressing human breast carcinoma cell lines was determined. Remarkably, all LBH-expressing tumor cell lines harbor unusually high percentages of CSC populations that are either CD44^{high}/CD24^{low} (>80% in MDA-MB231 and HCC1395 cell lines) or ALDH+ (>8% in HCC1187) (FIG. 11). In contrast, all LBH-negative BC cell lines have low or undetectable amounts of CD44^{high}/CD24^{low} CSCs. From these studies and data described herein demonstrating that LBH is specifically overexpressed in triple negative breast cancer (TNBC), which are enriched in CD44^{high}/CD24^{low} CSCs, it is concluded that LBH expression correlates with breast CSC phenotype.

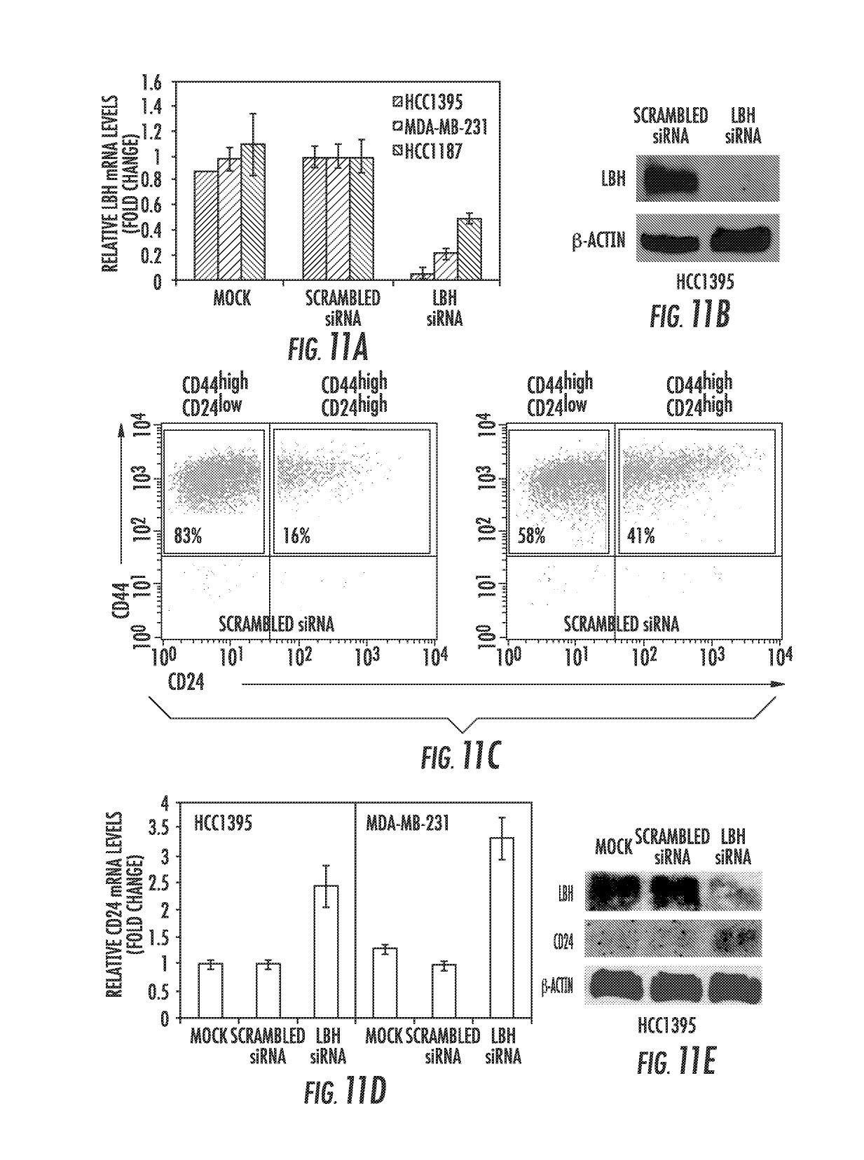

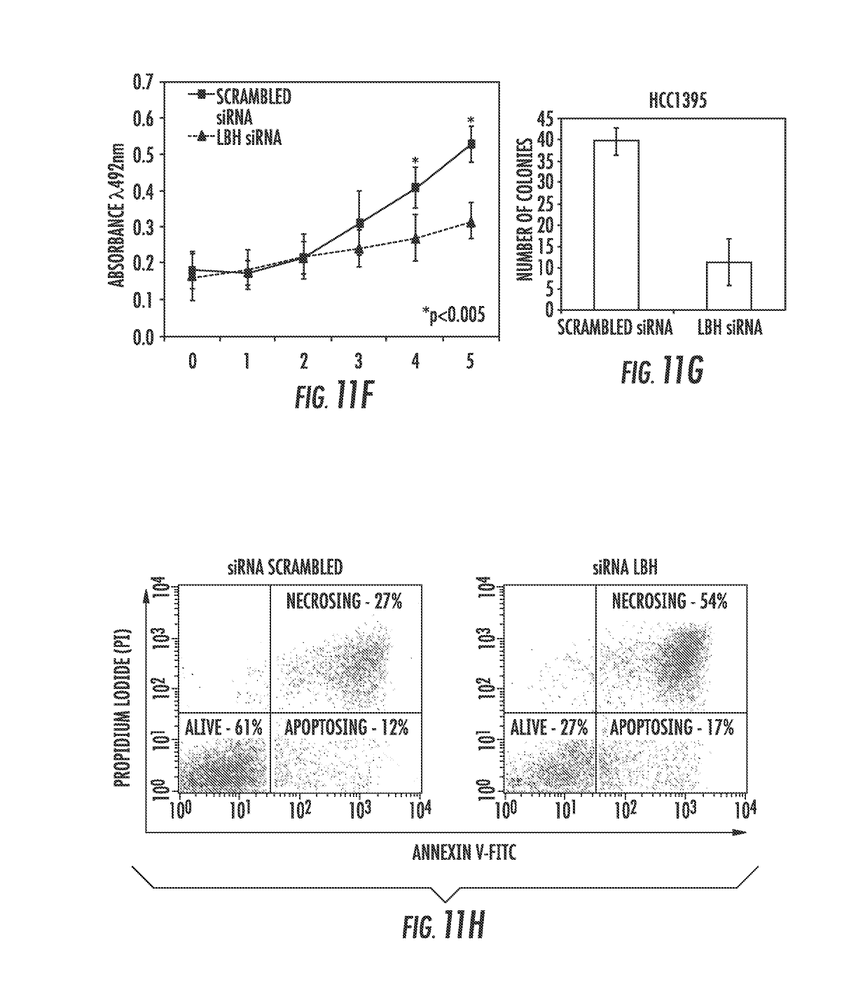

Immunohistochemistry was employed to determine LBH expression in triple-negative breast cancers. An IHC protocol on clinical specimen using an affinity-purified LBH antibody has been established. In a study involving over 8 TNBC samples, it was discovered that LBH protein is specifically overexpressed in a stem-like subgroup of TNBC that is highly metastatic and chemoresistant, the so-called metaplastic breast tumors (FIG. 10). Interestingly, although genetic mutations in WNT pathway genes in breast cancer are rare, metaplastic tumors frequently harbor mutations that lead to constitutive activation of the WNT stem cell self-renewal pathway, providing another link between LBH-WNT and breast cancer stem cells. Moreover, these studies showed that LBH is expressed in the basal-myoepithelial cell lineage, which harbors progenitor cells with increased repopulation ability in normal breast tissue (FIG. 10).

The consequences of siRNA-mediated ablation of LBH in human TNBC breast carcinoma cell lines on cell proliferation, apoptosis and cell motility were investigated. To explore whether LBH is required for the seemingly ‘stem-like’ nature of these TNBC cell lines, RNAi knockdown (KD) conditions were established to efficiently deplete LBH in these cell lines (FIGS. 11A, B). Strikingly, FACS analysis 9 days post siRNA transfection in over three independent experiments showed that KD of LBH drastically reduced (25%) the CD44^{high}/CD24^{low} CSC population (FIG. 11C), whereas it led to a reciprocal increase of CD44^{high}/CD24^{high} tumor cells suggesting that LBH depletion leads to acquisition of a more luminal tumor phenotype, which has a better prognosis. Moreover, expression levels of the luminal marker CD24 were significantly increased in HCC1395-LBH KD cells (FIGS. 11C, E). Next, cell growth and apoptosis rates in HCC1395 LBH-KD and control cells were analyzed to explore whether the observed shift to a more differentiated tumor phenotype could be due to a requirement of LBH function for CD44^{high}/CD24^{low} CSC survival. Indeed, reduced cell viability and anchorage-independent growth in soft agar was observed in LBH-KD cells (FIGS. 11F,G). This was attributed to increased cell death, as evaluated by Annexin V immunostaining (FIG. 11H). Even more remarkably, stable LBH KD using lentiviral transduction of HCC1395 cells with LBH-specific shRNA expression vectors resulted in nearly complete cell death of HCC1395 cells (FIGS. 11I, J). Thus, LBH appears to be required for the survival of the mostly CD44^{high}/CD24^{low} HCC1395 cells. To explore whether LBH affects tumor progression of TNBC cells, LBH was stably expressed in BT549 cells. This tumor line lacks LBH expression and has low tumorigenicity in vivo. As shown in FIG. 11L, BT549+LBH cells demonstrated a greater propensity to form colonies in soft agar compared to vector control cells, indicating LBH overexpression is oncogenic. The sequences of the LBH-specific shRNAs used in the experiments described herein are as follows: shRNA#1 CCGGGCTTGTAAACTGCGTAACAAACTCGAGTTTGTTACGCAGTTTACAAGCTTTTT G (SEQ ID NO:12) and shRNA#5 CCGGGCGAAGAGACAGCGAAAGAAACTCGAGTTTCTTTCGCTGTCTCTTCGCTTTTT G (SEQ ID NO:13).

To investigate the effects of LBH overexpression on tumor cell differentiation, stable ER-positive luminal MCF7 breast tumor cell lines expressing LBH were established and it was found that ectopic LBH expression alters cell proliferation and reduces ER expression. Moreover, ectopic expression of LBH in non-transformed HC11 mouse mammary epithelial cells blocked ER expression and luminal differentiation as measured by induction of the milk-protein β-casein upon treatment with lactogenic hormones. Collectively, the data demonstrate that LBH overexpression in TNBC plays a causal role in promoting the undifferentiated CSC phenotype and ER-negativity that is characteristic of these highly aggressive breast tumors. Since LBH transcription cofactor function is critically required for the survival of TNBC cells, pharmaceutical inhibition of LBH holds great promise for future treatment of TNBC.

The studies described herein demonstrate that LBH is a novel biomarker and therapeutic target for the most lethal form of breast cancer, called triple-negative or basal subtype breast cancer. Triple negative breast cancers account for ˜20% of all breast cancers, but rank 6th on the general cancer death statistics due to an unusual aggressive clinical course and current lack of specific treatment options. The key discovery that inhibition of LBH kills triple negative breast tumor cells, which are mostly cancer stem cells, or induces their differentiation, has a high impact and important implications for developing molecular target therapies for the treatment of triple-negative and metastatic, treatment-refractory breast cancers, for which there is currently no cure.

### Example 7

### LBH Overexpression in Colon Cancer

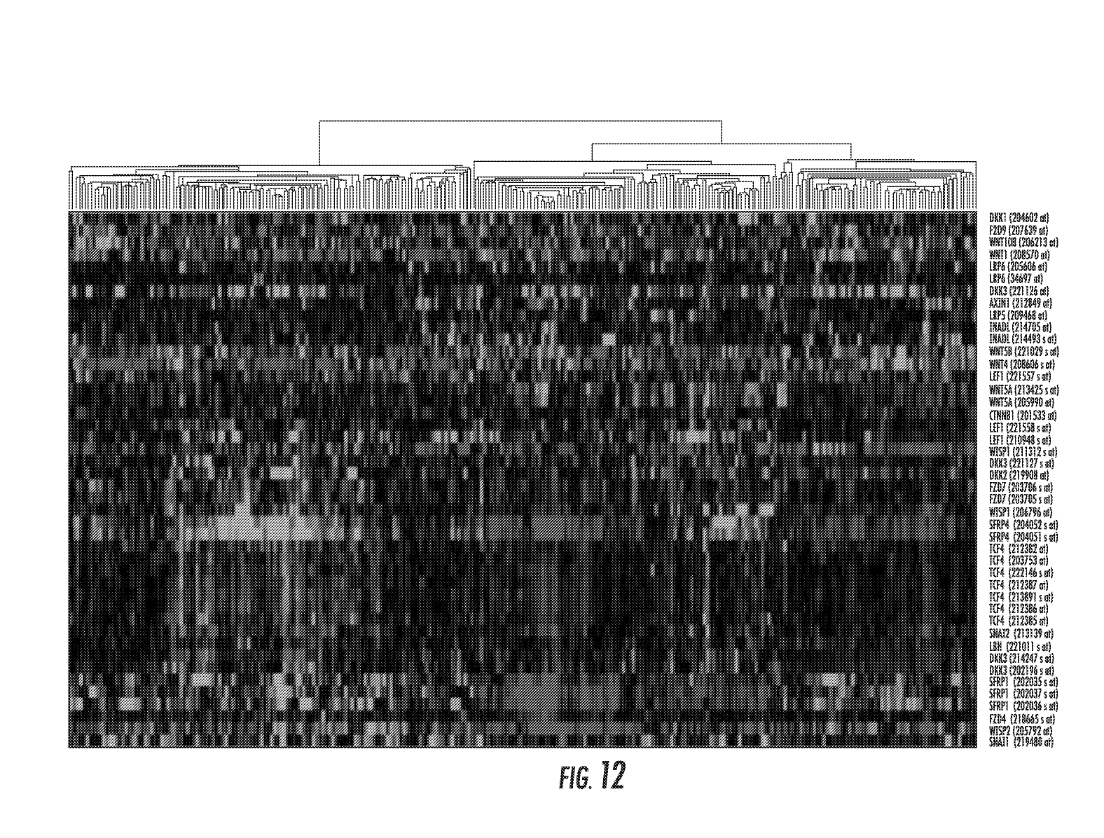

FIG. 12 shows a clinical association of LBH with WNT pathway activation in colon cancer. In this experiment, a Meta-analysis of 281 colon tumors from the Expo data set was performed and demonstrates that LBH (denoted by the arrow) is expressed in a subset of colon tumors. Like in breast cancer, LBH expression positively correlates with expression of a subset of Wnt target genes including TCF4, SFRP1, and DKK3. The Pearson correlation coefficients are shown to the right.

### Other Embodiments

Any improvement may be made in part or all of the compositions, kits, transgenic animals and method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. A composition comprising a therapeutically effective amount of an LBH inhibitor for inhibiting cancer cell growth in a subject having cancer cells and a pharmaceutically acceptable carrier.

2. The composition of claim 1, wherein the LBH inhibitor is LBH-specific siRNA.

3. The composition of claim 1, wherein the cancer cells are breast cancer cells.

4. The composition of claim 3, wherein the breast cancer cells are triple-negative breast cancer cells.

5. A composition comprising a therapeutically effective amount of WNT7a protein or nucleic acids encoding WNT7a protein for inhibiting cancer cell growth in a subject having cancer cells and a pharmaceutically acceptable carrier.

6. The composition of claim 5, wherein the cancer cells are breast cancer cells.

7. The composition of claim 6, wherein the breast cancer cells are triple-negative breast cancer cells.

8. A method of inhibiting growth of cancer cells, comprising contacting the cancer cells with a composition comprising a therapeutically effective amount for inhibiting cancer cell growth of at least one selected from the group consisting of: an LBH inhibitor, a WNT7a protein, and a nucleic acid encoding WNT7a protein, under conditions such that the cancer cells die or differentiate.

9. The method of claim 8, wherein the cancer cells are triple-negative breast cancer cells.

10. The method of claim 8, wherein the composition comprises an LBH inhibitor and a WNT7a protein or a nucleic acid encoding WNT7a protein.

11. A method of treating a subject having estrogen receptor negative basal-type breast cancer comprising: administering to the subject a composition comprising a pharmaceutical carrier and at least one of: an LBH inhibitor, a WNT7a protein, and a nucleic acid encoding WNT7a protein in an amount effective for inhibiting growth of estrogen receptor negative basal-type breast cancer cells in the subject.

12. The method of claim 11, wherein the composition comprises an LBH inhibitor and a WNT7a protein or a nucleic acid encoding WNT7a protein.

13. The method of claim 11, wherein the composition comprises an LBH inhibitor and the LBH inhibitor is LBH-specific siRNA.

14. A method of detecting the presence of cancer in a subject comprising:
(a) obtaining a biological sample from the subject;
(b) contacting the sample with at least one reagent that detects the presence of LBH expression;
(c) measuring the level of LBH expression in the biological sample; and
(d) correlating overexpression of LBH in the sample with the presence of cancer cells in the subject.

15. The method of claim 14, wherein the cancer is estrogen receptor negative basal-type breast cancer.

16. The method of claim 14, wherein the at least one reagent is an LBH-specific antibody.

17. The method of claim 14, wherein steps b) and c) are performed using real-time polymerase chain reaction (PCR) and the at least one reagent comprises a pair of LBH-specific primers.

18. A kit for detecting the presence of estrogen receptor-negative basal-type breast cancer in a subject, the kit comprising:
(a) at least one reagent for detecting the presence of LBH expression and quantitating the expression of LBH in a biological sample from the subject; and
(b) instructions for use.

19. The kit of claim 18, wherein the at least one reagent is an LBH-specific antibody.

20. The kit of claim 18, wherein the at least one reagent comprises a pair of LBH-specific primers.


SEQUENCE LISTING (13 sequences)

SEQ ID NO: 1 - protein, 105 aa, homo sapiens
MSIYFPIHCPDYLRSAKMTEVMMNTQPMEEIGLSPRKDGLSYQIFPDPSDFDRCCKLKDRLPSIVVEPTEGEVESGELRWPPEEFLVQEDEQDNCEETAKENKEQ

SEQ ID NO: 2 - protein, 349 aa, homo sapiens
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCK

SEQ ID NO: 3 - DNA, 2956 bp, homo sapiens
ggggctgagtgctcagtggagagcggggagttgtgtccaccttgccgacgtcgctagccgtggggctgtcctgggaaggcggacggcgagcgcccggtgtccgcactcggccgcctgccgtgcccgtctgcgcccgtgtcatcctcactcgggacgcagggaccgtttttaaatcacaggggcgtgtgtcagcctgccctaggacttcatgtctatatatttccccattcactgccccgactatctgagatcggccaagatgactgaggtgatgatgaacacccagcccatggaggagatcggcctcagcccccgcaaggatggcctttcctaccagatcttcccagacccgtcagattttgaccgctgctgcaaactgaaggaccgtctgccctccatagtggtggaacccacagaaggggaggtggagagcggggagctccggtggccccctgaggagttcctggtccaggaggatgagcaagataactgcgaagagacagcgaaagaaaataaagagcagtagagtccctgtggactcccatgggtcataccagccagcatctgttcctgaactgtgtttttcccatcatgacggaagaagagagtgagccgcaattgttctgaaaatgtcaaacgaggcttctgttttgcacctgcagatcaccgagttggttttcttttcttttcttgcctttttttttttttgaaatttgccgagcagtggagccctctgacaatttgcaaggccctctgagaaaggaagctgcttagagccagggggttagtgggtgaggggagcgagtgctgtttttgagatcattatctgaactcaggcagcctagtagaggcagtggtgggattccaatgggtcttggtgggtgggaggtggggcatgtgcaaagcaagcaaggaacatttggggtaagaaaacaaacatgaggcaaaagaaaaaatacatgtttttaagaaaacattgagcagagaactgcagccaggatgcgctcagcagacattcactctggctgctgggacatcagaaaacaaagtcttcatctctctctccagtttcacccaccccaccctttgctttcatttcaggtgtgttggtctatatgacagggaggagagtaaaggagagcaggagcaattggctgcctgcaaagccagctggaggtgaagtgcaggaaaggaaaggtcaccccattctactccatggcctctctgctcccagctgtggtaggctcacatagccagtgtgatcggtttttaagaggcagtgcttttcagcttttctccctgatatatccattttgcttcccagcactttttaggagtagtgagagcacttcctgcccttgttggaagccccagggtggacactcagcacgaaggtctctcccttaactgctgcccttccaagacttgctcccgagatggagtgggcgtggtcttccaggctggcccttccttctcctcaccgccaccttccctgccccagccccagcagccatgggtacatgggtccccagctcacctatggattcccgccagtctgcccagctgcagtactcacgccccatgggggatcttggtctgtttttcttgtgggagcctagtggagagcagacgtggctttttatgtgtcttgttggggaggtgacttgcatggtggggacaaggctgtcgtggcaaccttgggatcgagtttgagactaaaggatgtcatgagatccctggcttctccccatgttgttcccggacaagggcagaagggaggcatggcaagggacctctgctgtccttactcaacagtggtcctcatccctccccacctcccactgcttcctgcaagggcaccagttgtatgagaaagttggcctttggacttaggatttcttattgtagctaagagccatctgaagcagcaggttgcaggacaaatgcttcagtccgccgagagcagtaccgtgtggccaagaggtggactcagagccttccttgagctaaactcggccaaccaaggcacgcagcatgtcccctcaggtctccagtcagtccaggttgaccctcagttctggacgtgtgtatatagctgtatttaatacctcaaggtcattgtggctctggggatgccggggcaggaggacgagggtgcgctgtggacacagcagtccgcggaattccgttctgggaagccaatggtcgccggcaccccttgcttcctccctctgttgtctgcctgtgtgacacacatcaatggcaataacttcttccaactcctcgcagaagtgggagaggccggcagcctgcaccgagaggggctttcctctctcttgctccccgcttcgttctgttttggctgcagagagtggttcatccatactctcattccctcgcctccccttgtggacgggggtcttgccttttcaattcctgtgttttggtgtcttcccttatctgctaccctgaatcacctgtcctggtcttgctgtgtgatgggaacatgcttgtaaactgcgtaacaaatctactttgtgtatgtgtctgtttatgggggtggtttattatttttgctggtccctagaccactttgtatgaccgtttgcagtctgagcaggccaggggctgacagctaatgtcaggaccctcagcggtggagcctgctggggggacccagctgctcttggacaagtggctgagctcctatctggcctcctctttttttttttttcaagtaatttgtgtgtatttctaactgattgtattgaaaaaattcctagtatttcagtaaaaatgcctgttgtgagatgaacctcctgtaacttctatctgttcttttttgaggctcagggagaaactagcatttttttttttccaaactactttttgtcactgtgacagttgtaaataaagtttgaaaatgctttccactctgaaaaaaaaaaaaaaaaaa

SEQ ID NO: 4 - DNA, 1732 bp, HOMO SAPIENS
gaggggcgggggctggaggcagcagcgcccccgcactccccgcgtctcgcacacttgcaccggtcgctcgcgcgcagcccggcgtcgccccacgccgcgctcgctcctccctccctcctcccgctccgtggctcccgtgctcctggcgaggctcaggcgcggagcgcgcggacgggcgcaccgacagacggccccggggacgcctcggctcgcgcctcccgggcgggctatgttgattgccccgccggggccggcccgcgggatcagcacagcccggcccgcggccccggcggccaatcgggactatgaaccggaaagcgcggcgctgcctgggccacctctttctcagcctgggcatggtctacctccggatcggtggcttctcctcagtggtagctctgggcgcaagcatcatctgtaacaagatcccaggcctggctcccagacagcgggcgatctgccagagccggcccgacgccatcatcgtcataggagaaggctcacaaatgggcctggacgagtgtcagtttcagttccgcaatggccgctggaactgctctgcactgggagagcgcaccgtcttcgggaaggagctcaaagtggggagccgggaggctgcgttcacctacgccatcattgccgccggcgtggcccacgccatcacagctgcctgtacccagggcaacctgagcgactgtggctgcgacaaagagaagcaaggccagtaccaccgggacgagggctggaagtggggtggctgctctgccgacatccgctacggcatcggcttcgccaaggtctttgtggatgcccgggagatcaagcagaatgcccggactctcatgaacttgcacaacaacgaggcaggccgaaagatcctggaggagaacatgaagctggaatgtaagtgccacggcgtgtcaggctcgtgcaccaccaagacgtgctggaccacactgccacagtttcgggagctgggctacgtgctcaaggacaagtacaacgaggccgttcacgtggagcctgtgcgtgccagccgcaacaagcggcccaccttcctgaagatcaagaagccactgtcgtaccgcaagcccatggacacggacctggtgtacatcgagaagtcgcccaactactgcgaggaggacccggtgaccggcagtgtgggcacccagggccgcgcctgcaacaagacggctccccaggccagcggctgtgacctcatgtgctgtgggcgtggctacaacacccaccagtacgcccgcgtgtggcagtgcaactgtaagttccactggtgctgctatgtcaagtgcaacacgtgcagcgagcgcacggagatgtacacgtgcaagtgagccccgtgtgcacaccaccctcccgctgcaagtcagattgctgggaggactggaccgtttccaagctgcgggctccctggcaggatgctgagcttgtcttttctgctgaggagggtacttttcctgggtttcctgcaggcatccgtgggggaaaaaaaatctctcagagccctcaactattctgttccacacccaatgctgctccaccctcccccagacacagcccaggtccctccgcggctggagcgaagccttctgcagcaggaactctggacccctgggcctcatcacagcaatatttaacaatttattctgataaaaataatattaatttatttaattaaaaagaattcttccacaaaaaaaaaaaaaaaaaa

SEQ ID NO: 5 - DNA, 10 bp, homo sapiens
cctttgatct

SEQ ID NO: 6 - DNA, 10 bp, homo sapiens
cctttgaatc

SEQ ID NO: 7 - DNA, 10 bp, homo sapiens
tctttgatat

SEQ ID NO: 8 - DNA, 10 bp, homo sapiens
gctttgaaga

SEQ ID NO: 9 - DNA, 10 bp, homo sapiens
actttgtact

SEQ ID NO: 10 - DNA, 19 bp, artificial sequence
  synthetic construct
tcactgccccgactatctg

SEQ ID NO: 11 - DNA, 19 bp, artificial sequence
  synthetic construct
ggttccaccactatggagg

SEQ ID NO: 12 - DNA, 58 bp, artificial sequence
  synthetic construct
ccgggcttgtaaactgcgtaacaaactcgagtttgttacgcagtttacaagctttttg

SEQ ID NO: 13 - DNA, 58 bp, artificial sequence
  synthetic construct
ccgggcgaagagacagcgaaagaaactcgagtttctttcgctgtctcttcgctttttg